(19) United States
(12) Patent Application Publication
(10) Pub. No.: US 2018/0015075 A1
(43) Pub. Date: Jan. 18, 2018

(54) METHODS AND COMPOSITIONS FOR TREATMENT OF VENOUS MALFORMATION

(71) Applicant: Children's Hospital Medical Center, Cincinnati, OH (US)

(72) Inventors: Elisa Boscolo, Cincinnati, OH (US); Xian Li, Lexington, KY (US)

(21) Appl. No.: 15/649,786

(22) Filed: Jul. 14, 2017

Related U.S. Application Data
(60) Provisional application No. 62/362,184, filed on Jul. 14, 2016.

(51) Int. Cl. A61K 31/436; A61K 31/5025; A61K 31/496; A61K 31/506; A61K 9/00

(52) U.S. Cl. CPC A61K 31/436; A61K 31/506; A61K 9/0019; A61K 31/496; A61K 31/5025

(57) ABSTRACT

Methods of treating venous malformation (VM) are described. The described methods may include the steps of administering an mTOR inhibitor and an ABL kinase inhibitor to an individual in need thereof. Articles of manufacture comprising a container and a composition comprising the actives used in the disclosed methods are also described.

### CROSS REFERENCE TO RELATED APPLICATIONS

This application claims priority to and the benefit of U.S. Provisional Patent Application Ser. No. 62/362,184, filed Jul. 14, 2016, to Boscolo and Li, entitled “BCR-ABL Inhibitors to Treat Venous Malformations with Activated TIE2,” the contents of which are incorporated by reference in its entirety.

### GOVERNMENT SUPPORT CLAUSE

This invention was made with government support under HL117952 awarded by the National Institute of Health. The government has certain rights in the invention.

### BACKGROUND

Venous malformation (VM) is a slow-flow vascular anomaly which has an estimated incidence of approximately 1/10000 (1-3). VM forms compressible bluish lesions that can grow commensurately with the developing child causing disfiguration (3, 4). Pathologically, VM lesions are characterized by ecstatic endothelial cells (EC)-lined channels covered by rare and irregularly distributed smooth muscle cells (5). Expansion of VM lesions cause extensive disfigurement, organ dysfunction and chronic symptoms such as bleeding, oozing and pain (6, 7). VMs cause deformity, pain, and local intravascular coagulopathy, and they expand with time. Elevated D-dimer levels (>0.5 μg/ml) in VM patients can be associated with spontaneous thrombosis and Localized Intravascular Coagulopathy (LIC). To reduce the thrombosis events and pain caused by phleboliths, tailored compression garments and low molecular weight heparin are used (3, 4). Sclerotherapy, alone or in combination with surgery, is the standard first-line therapy. These interventional procedures have high complication rate and lesions recur (8).

### BRIEF SUMMARY

Methods of treating venous malformation (VM) are described. The described methods may include the steps of administering an mTOR inhibitor and an ABL kinase inhibitor to an individual in need thereof. Articles of manufacture comprising a container and a composition comprising the actives used in the disclosed methods are also described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. ABL kinase inhibition prevents murine VM lesion formation. (Panel A) Western blot analysis of non-transfected HUVECs (NT), HUVECs transfected with wild-type TIE2 (WT) or with mutant TIE2 (L914F) probed with indicated antibodies. (Panel B) Western blot analysis of HUVECs transfected with mutant TIE2 (L914F) probed with indicated antibodies. Cells were treated with DMSO (control) or 500 nM Ponatinib for 1 hour. (Panel C) Treatment scheme. HUVEC TIE2-L914F injected nude mice were treated with vehicle or Ponatinib (30 mg/kg/day, oral gavage) for 14 days. (Panel D) Lesion size measured by caliper every 2 days; Data expressed as mean±SD, t test (n=6 mice with 2 lesions/group). (Panel E) Representative images of lesions. Scale bar: 1 cm. (Panel F) Representative images of H&E stained sections from middle of the explant. Scale bar: 500 μm. (Panel G) Quantification of vessel area. Data expressed as single value for each lesion, mean and SD shown by horizontal bars, t test (n=6 mice with 2 lesions/group).

FIG. 2. Ponatinib combined with Rapamycin inhibits cell proliferation in vitro and VM lesion growth in vivo. (Panel A) HUVEC-TIE2-L914F were treated with different concentrations (from 10 μM to 0.03 μM) of ABL kinase inhibitors alone or in the presence of rapamycin at 10 nM for 72 h. Data expressed as mean±SD, n=4. (Panel B) Lesion size. HUVEC TIE2-L914F injected mice were treated by oral gavage every day with vehicle, Ponatinib (30 mg/kg), rapamycin (2 mg/kg), both or reduced dose (Ponatinib 20 mg/kg/day and rapamycin 1 mg/kg) for 15 days. Lesions were measured by caliper every 2 days. Data expressed as mean±SD, t test (n=8 mice with 2 lesions/group). (Panel C) Representative images of lesions. (Panel D) Lesion weight. Data expressed as single value for each lesion, mean and SD shown by horizontal bars, t test (n=8 mice with 2 lesions/group). (Panel E) Representative H&E stained sections. Scale bar: 500 μm. (Panel F) Quantification of vessel area. Data expressed as single value for each lesion, mean shown by horizontal bars, t test (n=8 mice with 2 lesions/group).

FIGS. 3A-3H. Ponatinib combined with Rapamycin induces murine VM lesion regression, even at reduced dose. (FIG. 3A) Treatment scheme. When average VM lesion size reached 100 mm² treatment was started (day 9) by oral gavage every day with vehicle, Ponatinib (30 mg/kg), rapamycin (2 mg/kg), combination and reduced dose combination (RD Combo, Ponatinib 20 mg/kg/day+rapamycin 1 mg/kg) for 14 days. (FIG. 3B) Waterfall plot of Log 10 of lesion size change (Day 23/Day 9). Data expressed as single values for each lesion (n=8 mice with 2 lesions/group). (FIG. 3C) Log 10 of lesion weight. Data expressed as single value for each lesion, mean shown by horizontal bars (n=8 mice with 2 lesions/group). (FIG. 3D) Representative H&E stained sections. Scale bar: 500 μm. (FIG. 3E) Quantification of vessel area. Data expressed as single values for each lesion, mean shown by horizontal bars, t test (n=8 mice with 2 lesions/group). (FIG. 3F) Representative cleaved caspase-3 and Ki-67 staining of lesion sections. Scale bar: 500 μm. (FIG. 3G) Quantification of cleaved caspase-3 events and (FIG. 3H) Quantification of Ki-67 events. Data expressed as mean±SD, t test (n=5).

FIGS. 4A-4D. Long-term combination treatment of Ponatinib and rapamycin in VM murine model. (FIG. 4A) Western blot analysis of VM lesions probed with indicated antibodies. When average VM lesion size in each group (8 mice per group) reached 130 mm̂2 (day 12) after HUVEC-TIE2-L914F injection, treatment was started by oral gavage every day with vehicle, Ponatinib (30 mg/kg), rapamycin (2 mg/kg), combination and reduced dose combination (RD Combo, Ponatinib 20 mg/kg/day+rapamycin 1 mg/kg) for 28 days. Then lesions were dissected at day 41 and analyzed by Western blotting (FIG. 4B) Densitometric analysis of p-AKT473 Western blot bands relative to total AKT. Data are normalized to vehicle group and were expressed as mean±SD. (FIG. 4C) Quantification of lesion weight and mouse body net weight at day 41. Data expressed as mean±SD, t test (n=8 mice with 2 lesions/group). (FIG. 4D) Graphs of blood chemical profile and complete blood count (CBC) in vehicle, combination and RD combo group. ALT (Alanine aminotransferase), AST (Aspartate aminotransferase), BUN (Blood Urea Nitrogen), HBG (Hemoglobin), RBC (Red Blood Cells). Red dashed lines indicated reference value and grey square-holes show values in normal nude mice without cell injection. Data expressed as single values for each lesion, mean shown by horizontal bars, t-test (n=8 mice/group).

FIGS. 5A-5F. Ponatinib combined with Rapamycin enhances apoptosis in HUVEC-TIE2-L914F. (FIG. 5A) Flow cytometric analysis of apoptosis. HUVEC TIE2-L914F cells were treated with Vehicle (DMSO), 100 nM Ponatinib, 10 nM rapamycin or combination for 72 hours. (FIG. 5B) Quantification of apoptotic cell populations. Data expressed as mean±SD, n=2 independent experiments. (FIG. 5C) Representative images of cell cycle analysis. HUVEC TIE2-L914F cells were treated with Vehicle, 100 nM Ponatinib, 10 nM rapamycin or combination for 48 hours. (FIG. 5D) Quantification of G0-G1 cell cycle cell population. Data expressed as mean±SD, n=2 independent experiments. (FIG. 5E) Representative images of cell migration. HUVEC TIE2-L914F cells were treated with Vehicle, 100 nM Ponatinib, 10 nM rapamycin or combination for 10 hours after the scratch was performed. (FIG. 5F) Quantification of migration speed. Data expressed as mean±SD, n=2 independent experiments.

FIG. 6. Ponatinib combined with Rapamycin induced channel regression in fibrin gel bead assay. (Panel A) Treatment scheme and representative images. HUVEC-TIE2-L914F-GFP cells were treated with Vehicle, 100 nM Ponatinib, 10 nM rapamycin or combination at day 1 when beads were coated with cells and embedded in the fibrin gel. (Panel B) Quantification of tube/channel area. Data expressed as mean±SD, n=2 independent experiment. (Panel C) Treatment scheme of channel regression and representative images. HUVEC TIE2-L914F-GFP cells were treated with Vehicle, 100 nM Ponatinib, 10 nM rapamycin or combination at Day 8 when tube networks were established. (Panel D) Quantification of tube/channel area change from day 8 to day 14. Data expressed as mean±SD, n=2 independent experiments.

FIG. 7. Ponatinib combined with Rapamycin inhibits AKT and ERK signaling. (Panel A) Phosphorylation changes of 45 kinase sites in HUVEC-TIE2-L914F(L914F) relative to HUVEC-TIE2-WT(WT). HUVEC-TIE2-WT and HUVEC-TIE2-L914F cell lysates were analyzed by Phospho-Kinase Array Kit and then quantified. (Panel B) Phosphorylation changes of top 10 target sites (from Panel A) in response to Ponatinib, rapamycin or combination treatment. HUVEC-TIE2-L914F were treated by vehicle (DMSO), 100 nM Ponatinib, 10 nM rapamycin or combination for 48 hours. (Panel C) Western blotting analysis of HUVEC-TIE2-L914F with indicated antibodies. Cells were treated with DMSO (Control), 100 nM Ponatinib, 10 nM rapamycin or both for indicated time.

FIG. 8. Ponatinib combined with Rapamycin induces VM lesion regression in PDX model. (Panel A) Representative images of VM-patient-derived EC (VM-EC) cell morphology and stained for CD31 (green) and DAPI (blue). CD31 staining. (Panel B) Sanger sequencing results of normal HUVEC and VM-EC. (Panel C) Overview of VM-EC lesions at day 32. When average VM lesion size reached 60 mm² (day 18), treatment was started by oral gavage with vehicle, rapamycin (2 mg/kg) and combination (Ponatinib 30 mg/kg/day+rapamycin 2 mg/kg) for 14 days. 8 mice in vehicle and combination treatment group and 7 mice in rapamycin treatment group each mouse with 2 lesions. (Panel B) Waterfall plot of log 10 of lesion size change (Day 32/Day 18). Data expressed as single values for each lesion. (Panel D) Representative images of H&E stained sections. Scale bar: 500 μm. (Panel E) Quantification of vessel area. Data expressed as single values for each lesion, mean shown by horizontal bars, t-test (n=8 mice with 2 lesions/group in vehicle and combination treatment group and n=7 mice with 2 lesions/group in rapamycin treatment group).

FIG. 9. Mouse weight in Ponatinib treated mice. Mouse eight was measured every 2 days until day 16. Data expressed as mean±SD, t-test (n=6 mice/group).

FIG. 10. Combination treatment with Ponatinib and rapamycin in the VM murine model. (Panel A) Lesion size of each group. HUVEC TIE2-L914F injected nude mice were treated by oral gavage every day with vehicle, Ponatinib (30 mg/kg), rapamycin (2 mg/kg) or both for 15 days. Lesion size measured by caliper every 2 days until day 16. Data expressed as mean±SD, t-test (n=8 mice with 2 lesions/group). (Panel B) Mouse weight measured every 2 days until day 16. Data expressed as mean±SD, t-test (n=8 mice/group). (Panel C) Overview of lesions. Scale bar: 1 cm. (Panel D) Quantification of lesion weight. Data expressed as single values for each lesion, mean shown by horizontal bars, t-test (n=8 mice with 2 lesions/group). (Panel E) Representative H&E stained sections. Scale bar: 500 μm. (Panel F) Quantification of vessel area. Data expressed as single values for each lesion, mean shown by horizontal bars, t-test (n=8 mice with 2 lesions/group)

FIG. 11. Comparison of different reduced dose combination treatments in the VM murine model. (Panel A) Lesion size of each group.

HUVEC TIE2-L914F injected mice were treated by oral gavage every day with vehicle, Ponatinib (30 mg/kg), rapamycin (2 mg/kg) or reduced dose1 (RD1 Combo, Ponatinib 20 mg/kg/day and rapamycin 1 mg/kg) or reduced dose2 (RD2 Combo, Ponatinib 10 mg/kg/day and rapamycin 0.5 mg/kg) for 15 days. Lesion size measured by caliper every 2 days until day 16. Data expressed as mean±SD, t-test (n=8 mice with 2 lesions/group). (Panel B) Mouse weight measured every 2 days until day 16. Data expressed as mean±SD, t-test (n=8 mice/group). (Panel C) Overview of lesions. (Panel D) Quantification of lesion weight. Data expressed as single values for each lesion, mean shown by horizontal bars, t-test (n=8 mice with 2 lesions/group). (Panel E) Representative H&E stained sections. Scale bar: 500 μm. (Panel F) Quantification of vessel area. Data expressed as single values for each lesion, mean shown by horizontal bars, t-test (n=8 mice with 2 lesions/group).

FIG. 12. Reduced dose combination treatment in the VM murine model.

FIG. 13. Ponatinib combined with rapamycin induced lesion regression, even at reduced dose. (Panel A) Lesion size of each group. When average VM lesion in each group reached 100 mm2 (day 9), HUVEC TIE2-L914F injected mice were treated by oral gavage every day with vehicle, Ponatinib (30 mg/kg), rapamycin (2 mg/kg), both (Combination) or reduced dose (RD Combo) (Ponatinib 20 mg/kg/day and rapamycin 1 mg/kg) for 14 days. Lesion size measured by caliper every 2 days. Data expressed as mean±SD, t-test (n=8 mice with 2 lesions/group). (Panel B) Quantification of mice net weight. Mice and lesion weight of each mice were measured at day 23, then mice net weight calculated. Data expressed as mean±SD, t-test (n=8 mice/group).

FIG. 14. Long-term combination treatment of Ponatinib with rapamycin in murine VM model. (Panel A) Treatment scheme. When average VM lesion in each group reached 130 mm2 (day 12), HUVEC TIE2-L914F injected mice were treated by oral gavage every day with vehicle, Ponatinib (30 mg/kg), rapamycin (2 mg/kg), both (Combination) or reduced dose (RD Combo) (Ponatinib 20 mg/kg/day and rapamycin 1 mg/kg) for 28 days. (Panel B) Waterfall plot of Log 10 of lesion size change (Day 41/Day 12). Red lines indicate −50% regression. Data expressed as single value for each lesion (n=8 mice with 2 lesions/group, one lesion in vehicle group was broken at day 34 and is not shown). (Panel C) Lesion size of each group. Data expressed as mean±SD, t-test (n=8 mice/group, one lesion in vehicle group was broken at day 34 and is not shown).

FIG. 15. Combination treatment with Ponatinib and rapamycin reduces cleaved caspase-3. (Panel A) Representative images of cleaved caspase-3 staining. (Panel B) Quantification of the ratio of cleaved caspase-3 events to nuclei. HUVEC TIE2-L914F cells were treated with DMSO, 300 nM Ponatinib, 10 nM rapamycin or both for 72 hours. Data expressed as mean±SD, t-test (n=4).

FIG. 16. Fibrin gel bead assay. (Panel A) Representative 2D images and 3D structural model of tube networks formed by HUVEC-GFP (left) or HUVEC-TIE2-L914F-GFP (right). (Panel B) Representative 2D images of HUVEC-TIE2-L914F-GFP tube network formation taken on the same field every 2 days from day 1 to 11. (Panel C) Representative images of HUVEC-TIE2-L914F-GFP tube network regression taken on the same field every other day from day 8 to 14.

FIG. 17. Quantification of Phospho-Kinase Array of treated HUVEC-TIE2-L914F. Image of Phospho-assay results. HUVEC-TIE2-L914F cells were treated with DMSO, 10 nM rapamycin, 100 nM Ponatinib or both for 48 hours and then analysis with Image Lab.

FIG. 18. Mouse body weight in VM PDX model. Mouse body weight was measured every 2 days for the duration of the treatment, from day 18 to 32. Data expressed as mean±SD, t-test (n=8 mice/group in vehicle and combination treatment group and n=7 mice/group in rapamycin treatment group).

FIG. 19. Schematic model of combination therapy in the treatment of VM. A TIE2 L914F mutation in a small subset of EC is sufficient to induce the formation of Venous Malformation (VM). Mutant VM EC show constitutive activation of TIE2 and downstream AKT and cABL (novel finding by Applicant), and drive lumen enlargement that leads to massive blood vessel expansion. In this manuscript, Applicant show that monotherapy treatment with Rapamycin or Ponatinib results in blood vessel stabilization (no expansion), moreover when Ponatinib and rapamycin are combined VM lesion undergo significant regression in size.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms and expressions used herein have the ordinary meaning as is accorded to such terms and expressions with respect to their corresponding respective areas of inquiry and study except where specific meanings have otherwise been set forth herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, for example, within 5-fold, and or for example, within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The term “therapeutically effective amount,” as used herein, refers to any amount of a compound which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The terms “treat,” “treating” or “treatment,” as used herein, refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

The term “pharmaceutically acceptable,” as used herein, refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “pharmaceutically acceptable salt,” as used herein, refers to a formulation of a compound that does not cause significant irritation to an organism to which it is administered and does not abrogate the biological activity and properties of the compounds described herein.

The terms “composition” or “pharmaceutical composition,” as used herein, refers to a mixture of at least one compound or antibody as disclosed herein, with at least one and optionally more than one other pharmaceutically acceptable chemical components, such as carriers, stabilizers, diluents, dispersing agents, suspending agents, thickening agents, and/or excipients.

The term “carrier” applied to pharmaceutical compositions of the disclosure refers to a diluent, excipient, or vehicle with which an active compound (e.g., dextromethorphan) is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like.

Venous malformation (VM) arises from developmental defects of the vasculature and is characterized by massively enlarged and tortuous venous channels. Lesions grow commensurately causing deformity, chronic swelling, obstruction of vital structures, bleeding and pain. Most VMs are associated with the activating endothelial cell tyrosine kinase receptor TIE2 mutation L914F. Therapeutic options for VM are limited and ineffective while target therapy such as mTOR inhibitor rapamycin shows moderate efficacy.

In one aspect, a method of treating venous malformation (VM), comprising the step of administering an mTOR inhibitor and an ABL kinase inhibitor to an individual in need thereof is disclosed. In one aspect, the mTOR inhibitor may be selected from rapamycin, 42-[3-Hydroxy-2-(hydroxymethyl)-2-methylpropanoate]-rapamycin, 42-O-(2-Hydroxyethyl)-rapamycin, or a combination thereof. In one aspect, the mTOR inhibitor may be rapamycin.

TABLE
mTOR inhibitors

| | | | Molecular | |
|---|---|---|---|---|
| Drug | Chemical name | Tradename | Formula | Supplier |
| Sirolimus | Rapamycin | Rapamune | C₅₁H₇₉NO₁₃ | Pfitzer |
| Temsirolimus | 42-[3-Hydroxy-2- | Torisel | C₅₆H₈₇NO₁₆ | Wyeth |
| | (hydroxymethyl)-2- | | | Pharmaceutical |
| | methylpropanoate]- | | | (Pfitzer) |
| | rapamycin | | | |
| Everolimus | 42-O-(2- | Afinitor | C₅₃H₈₃NO₁₄ | Novartis |
| | Hydroxyethyl)- | Zortress | | |
| | rapamycin | Votubia | | |

In one aspect, the ABL kinase inhibitor may be selected from 4-[(4-Methyl-1-piperazinyl)methyl]-N-[4-methyl-3-[[4-(3-pyridinyl)-2-pyrimidinyl]amino]-phenyl]benzamide methanesulfonate, 3-(2-imidazo[1,2-b]pyridazin-3-ylethynyl)-4-methyl-N-[4-[(4-methyl-1-piperazinyl)methyl]-3-(trifluoromethyl)phenyl]-benzamide, 4-Methyl-N-[3-(4-methyl-1H-imidazol-1-yl)-5-(trifluoromethyl)phenyl]-3-[[4-(3-pyridinyl)-2-pyrimidinyl]amino]-benzamide, 4-((2,4-Dichloro-5-methoxyphenyl)amino)-6-methoxy-7-(3-(4-methyl-1-piperazinyl)propoxy)3-quinolinecarbonitrile, N-(2-Chloro-6-methylphenyl)-2-[[6-[4-(2-hydroxyethyl)-1-piperazinyl]-2-methyl-4-pyrimidinyl]amino]-5-thiazolecarboxamide.

TABLE
ABL kinase inhibitors

| Drug | Chemical name | Tradename | Molecular Formula | Supplier |
|---|---|---|---|---|
| Imatinib | 4-[(4-Methyl-1- | Gleevec | C₂₉H₃₁N₇O•CH₄SO₃ | Novartis |
| | piperazinyl)methyl]-N- | Imatinib | | |
| | [4-methyl-3-[[4-(3- | Mesylate | | |
| | pyridinyl)-2- | | | |
| | pyrimidinyl]amino]- | | | |
| | phenyl]benzamide | | | |
| | methanesulfonate | | | |
| Ponatinib | 3-(2-imidazo[1,2- | Iclusig | C₂₉H₂₇F₃N₆O | ARIAD |
| | b]pyridazin-3- | | | Pharmaceutical |
| | ylethynyl)-4-methyl-N- | | | |
| | [4-[(4-methyl-1- | | | |
| | piperazinyl)methyl]-3- | | | |
| | (trifluoromethyl)phenyl]- | | | |
| | benzamide | | | |
| Nilotinib | 4-Methyl-N-[3-(4- | Tasigna | C₂₈H₂₂F₃N₇O | Novartis |
| | methyl-1H-imidazol-1- | | | |
| | yl)-5- | | | |
| | (trifluoromethyl)phenyl]- | | | |
| | 3-[[4-(3-pyridinyl)-2- | | | |
| | pyrimidinyl]amino]- | | | |
| | benzamide | | | |
| Bosutinib | 4-((2,4-Dichloro-5- | Bosulif | C₂₆H₂₉Cl₂N₅O₃ | Pfizer |
| | methoxyphenyl)amino)- | | | |
| | 6-methoxy-7-(3-(4- | | | |
| | methyl-1- | | | |
| | piperazinyl)propoxy)3- | | | |
| | quinolinecarbonitrile | | | |
| Dasatinib | N-(2-Chloro-6- | Sprycel | C₂₂H₂₆CIN₇O₂S | Bristol- |
| | methylphenyl)-2-[[6-[4- | | | Myers |
| | (2-hydroxyethyl)-1- | | | Squibb |
| | piperazinyl]-2-methyl-4- | | | |
| | pyrimidinyl]amino]-5- | | | |
| | thiazolecarboxamide | | | |

In one aspect, the mTOR inhibitor and the ABL kinase inhibitor may be administered sequentially, or in other aspects, simultaneously, to an individual in need thereof. In one aspect, the mTOR inhibitor may be administered to an individual daily, and the ABL kinase inhibitor may be administered to an individual weekly.

In one aspect, the mTOR inhibitor may be administered at a dose of from about 0.8 mg/m2, and the ABL Kinase inhibitor may be administered at a dose of from about 15 mg/m² to about 45 mg/m².

In one aspect, the rapamycin may be administered at a dose of from about 3 mg/m2/day to about 6 mg/m2/day, and the ABL kinase inhibitor may be administered at a dose of from about 30 to about 90 mg/m2 daily.

In one aspect, the mTOR inhibitor and the ABL kinase inhibitor may be administered in a single composition.

In one aspect, the mTOR inhibitor and the ABL kinase inhibitor may be administered in an amount sufficient to promote cell apoptosis in a venous malformation lesion and/or promote vascular channel regression.

In one aspect, the mTOR inhibitor and the ABL kinase inhibitor may be administered in an amount and for a duration sufficient to reduce a venous malformation lesion size and/or weight.

In one aspect, the mTOR inhibitor and the ABL kinase inhibitor are administered in an amount and for a duration sufficient to reduce the number of ki-67 expressing proliferative cells as compared to pre-treatment levels of ki-67 expressing proliferative cells.

The method of claim 1, wherein said venous malformation may be inherited cutaneomucosal venous malformation (VMCM) or blue rubber bleb nevus syndrome (BRBNS).

In one aspect, a method of promoting VM lesion regression and/or reducing VM lesion expansion in an individual in need thereof is disclosed. In this aspect, the method may comprise the step of administering an mTOR inhibitor and an ABL kinase inhibitor. In this aspect, the mTOR inhibitor and ABL kinase inhibitor are administered orally, intravenously, intralesionally, or a combination thereof. In one aspect, the mTOR inhibitor and ABL kinase inhibitor may be administered by direct lesional injection.

In one aspect, a method of normalizing hemoglobin levels and erythrocyte number in an individual having VM lesions, comprising the step of administering an mTOR inhibitor and an ABL kinase inhibitor, is disclosed, comprising administering an mTOR inhibitor and ABL kinase inhibitor as described herein.

In one aspect, an article of manufacture is disclosed. The article of manufacture may comprise a container comprising a label; and a first composition comprising an mTOR inhibitor or a pharmaceutically acceptable salt thereof and a pharmaceutically acceptable carrier, a second composition comprising an ABL kinase inhibitor or a pharmaceutically acceptable salt thereof and a pharmaceutically acceptable carrier, wherein the label indicates that said first composition and said second composition are to be administered to an individual having or at risk of developing VM lesions. In one aspect, the first and second composition are provided as a single composition. In a further aspect, the article of manufacture may further comprise a means for delivery of said composition to an individual.

Dosage

In one aspect, an agent disclosed herein may be present in an amount of from about 0.5% to about 95%, or from about 1% to about 90%, or from about 2% to about 85%, or from about 3% to about 80%, or from about 4%, about 75%, or from about 5% to about 70%, or from about 6%, about 65%, or from about 7% to about 60%, or from about 8% to about 55%, or from about 9% to about 50%, or from about 10% to about 40%, by weight of the composition.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, intralesional, or intramuscular forms all utilizing dosage forms well known to those of ordinary skill in the pharmaceutical arts. The compositions may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A dosage regimen will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a symptom or effect of a muscle contracture can be readily determined by an ordinarily skilled physician

The pharmaceutical compositions may include suitable dosage forms for oral, parenteral (including subcutaneous, intramuscular, intradermal and intravenous), transdermal, sublingual, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. Oral preparations include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. For topical or nasal administration, penetrants or permeation agents that are appropriate to the particular barrier to be permeated are used in the formulation. Such penetrants are generally known in the art. The pharmaceutical compositions are prepared by conventional techniques appropriate to the desired preparation containing appropriate amounts of the active ingredient, that is, one or more of the disclosed active agents or a pharmaceutically acceptable salt thereof according to the invention.

The dosage of an agent disclosed herein used to achieve a therapeutic effect will depend not only on such factors as the age, weight and sex of the patient and mode of administration, but also on the degree of inhibition desired and the potency of an agent disclosed herein for the particular disorder or disease concerned. It is also contemplated that the treatment and dosage of an agent disclosed herein may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

Kits

Kits are also provided. In one aspect, a kit may comprise or consist essentially of agents or compositions described herein. The kit may be a package that houses a container which may contain a composition comprising an oxime or pharmaceutically acceptable salt thereof as disclosed herein, and also houses instructions for administering the agent or composition to a subject. In one aspect, a pharmaceutical pack or kit is provided comprising one or more containers filled with one or more composition as disclosed herein. Associated with such container(s) can be various written materials such as instructions for use, or a notice in the form prescribed by a governmental agency regulating the manufacture, use or sale of pharmaceuticals or biological products, which notice reflects approval by the agency of manufacture, use, or sale for human administration.

As there may be advantages to mixing a component of a composition described herein and a pharmaceutically acceptable carrier, excipient or vehicle near the time of use, kits in which components of the compositions are packaged separately are disclosed. For example, the kit can contain an active ingredient in a powdered or other dry form in, for example, a sterile vial or ampule and, in a separate container within the kit, a carrier, excipient, or vehicle, or a component of a carrier, excipient, or vehicle (in liquid or dry form). In one aspect, the kit can contain a component in a dry form, typically as a powder, often in a lyophilized form in, for example, a sterile vial or ampule and, in a separate container within the kit, a carrier, excipient, or vehicle, or a component of a carrier, excipient, or vehicle. Alternatively, the kit may contain a component in the form of a concentrated solution that is diluted prior to administration. Any of the components described herein, any of the carriers, excipients or vehicles described herein, and any combination of components and carriers, excipients or vehicles can be included in a kit.

Optionally, a kit may also contain instructions for preparation or use (e.g., written instructions printed on the outer container or on a leaflet placed therein) and one or more devices to aid the preparation of the solution and/or its administration to a patient (e.g., one or a plurality of syringes, needles, filters, tape, tubing (e.g., tubing to facilitate intravenous administration) alcohol swabs and/or the Band-Aid® applicator). Compositions which are more concentrated than those administered to a subject can be prepared. Accordingly, such compositions can be included in the kits with, optionally, suitable materials (e.g., water, saline, or other physiologically acceptable solutions) for dilution. Instructions included with the kit can include, where appropriate, instructions for dilution.

In other embodiments, the kits can include pre-mixed compositions and instructions for solubilizing any precipitate that may have formed during shipping or storage. Kits containing solutions of one or more of the aforementioned active agents, or pharmaceutically acceptable salts thereof, and one or more carriers, excipients or vehicles may also contain any of the materials mentioned above (e.g., any device to aid in preparing the composition for administration or in the administration per se). The instructions in these kits may describe suitable indications (e.g., a description of patients amenable to treatment) and instructions for administering the solution to a patient.

### Examples

To identify novel therapeutical targets, Applicant performed unbiased screening of FDA-approved drugs in human umbilical vein endothelial cells expressing the TIE2-L914F mutation (HUVEC-TIE2-L914F). Applicant identified three ABL kinase inhibitors which caused significant cell proliferation inhibition in HUVEC-TIE2-L914F. c-ABL is a common target of these inhibitors, and here, Applicant are the first to report that c-ABL is highly phosphorylated downstream of TIE2-L914F. Furthermore, ABL kinase inhibitor Ponatinib, when combined with rapamycin, showed a synergistic anti-proliferative effect in vitro by promoting cell apoptosis and leading to significant vascular channel regression in a 3D fibrin gel assay. In vivo, treatment with a reduced dose of the drug combination Ponatinib and rapamycin triggered significant lesion regression in a murine and patient-derived xenograft model of VM. Minimal side effects were recorded. Analysis of drug combination mechanism showed enhanced AKT inhibition and reduced ERK activity due to rapamycin treatment. This is the first report for the use of Ponatinib and rapamycin combination for the treatment of VM.

TABLE 1
Unbiased cell-based drug screening assay results.
This table includes proliferation inhibition rates of 119 FDA approved drugs
at 20 uM in HUVEC-TIE2-1914F.

| | | | | | | | P value | | |
|---|---|---|---|---|---|---|---|---|---|
| | | | | | | | (compare | P value | |
| DRUG | Average proliferation | | | | | | L914F | (compare | |
| NAME | Inhibition rate (%) | | | | | | with | L914F | |
| (USAN) | L914F | | HUVEC | | WT | | HUVEC) | with WT) | Targets |
| Bortezomib | 81.02 ± | 0.89 | 81.03 ± | 5.14 | 69.51 ± | 7.04 | 1.000 | 0.065 | proteasome |
| | | | | | | | | | inhibitor |
| Enzalutamide | 80.92 ± | 1.44 | 82.91 ± | 2.91 | 66.47 ± | 9.64 | 0.346 | 0.079 | androgen |
| | | | | | | | | | receptor |
| | | | | | | | | | antagonist drug |
| Ixabepilone | 79.66 ± | 3.72 | 74.27 ± | 3.46 | 60.18 ± | 5.63 | 0.116 | 0.004 | antimicrotubules |
| Crizotinib | 78.52 ± | 3.35 | 63.84 ± | 1.51 | 64.75 ± | 11.63 | 0.002 | 0.130 | ALK and ROS1 |
| | | | | | | | | | inhibitor |
| Carfilzomib | 76.82 ± | 5.78 | 69.65 ± | 10.70 | 65.03 ± | 6.67 | 0.358 | 0.061 | proteasome |
| | | | | | | | | | inhibitor |
| Ceritinib | 76.61 ± | 1.43 | 12.43 ± | 13.48 | 15.47 ± | 17.03 | 0.004 | 0.008 | ALK inhibitor |
| Ponatinib | 75.54 ± | 2.74 | 26.65 ± | 14.27 | 50.75 ± | 12.14 | 0.008 | 0.035 | tyrosine kinase |
| | | | | | | | | | inhibitor |
| | | | | | | | | | (BCR-Abl) |
| Omacetaxine- | 74.10 ± | 2.26 | 79.78 ± | 1.33 | 67.59 ± | 6.99 | 0.014 | 0.207 | protein |
| mepesuccinate | | | | | | | | | translation |
| | | | | | | | | | inhibitor |
| Cabozantinib | 73.71 ± | 6.62 | 32.03 ± | 6.09 | 58.30 ± | 5.03 | 0.000 | 0.020 | multi-targeted |
| | | | | | | | | | RTK inhibitor |
| | | | | | | | | | (c-Met, VEGFR) |
| Mitoxantrone | 73.58 ± | 1.67 | 80.34 ± | 3.66 | 69.91 ± | 8.73 | 0.041 | 0.522 | type II |
| | | | | | | | | | topoisomerase |
| | | | | | | | | | inhibitor |
| Daunorubicin | 70.03 ± | 4.09 | 75.98 ± | 5.08 | 57.27 ± | 2.00 | 0.168 | 0.007 | antimetabolites |
| hydrochloride | | | | | | | | | |
| Mitomycin | 69.37 ± | 3.23 | 69.46 ± | 8.38 | 53.54 ± | 7.03 | 0.986 | 0.022 | chemotherapeutic |
| | | | | | | | | | agents |
| Topotecan | 68.43 ± | 3.07 | 77.50 ± | 2.36 | 63.84 ± | 9.29 | 0.008 | 0.467 | topoisomerase |
| hydrochloride | | | | | | | | | inhibitor |
| Vandetanib | 66.66 ± | 5.55 | 57.75 ± | 11.62 | 48.98 ± | 3.14 | 0.014 | 0.006 | multi-targeted |
| | | | | | | | | | RTK inhibitor |
| | | | | | | | | | (VEGFR, EGFR, |
| | | | | | | | | | RET) |
| Epirubicin | 64.66 ± | 1.97 | 74.39 ± | 4.94 | 54.67 ± | 3.51 | 0.035 | 0.009 | antimetabolites |
| hydrochloride | | | | | | | | | |
| Doxorubicin | 64.48 ± | 1.42 | 73.23 ± | 4.36 | 55.44 ± | 3.18 | 0.034 | 0.010 | antimetabolites |
| hydrochloride | | | | | | | | | |
| Teniposide | 63.88 ± | 6.46 | 60.03 ± | 3.71 | 49.87 ± | 10.98 | 0.414 | 0.117 | chemotherapeutic |
| | | | | | | | | | agent |
| Dactinomycin | 60.39 ± | 2.37 | 66.96 ± | 5.62 | 45.02 ± | 6.55 | 0.135 | 0.021 | inhibit |
| | | | | | | | | | transcription |
| Plicamycin | 60.14 ± | 2.17 | 65.66 ± | 4.17 | 47.75 ± | 4.92 | 0.104 | 0.015 | RNA synthesis |
| | | | | | | | | | inhibitor |
| Temsirolimus | 58.26 ± | 9.44 | 41.50 ± | 5.39 | 31.50 ± | 5.07 | 0.047 | 0.009 | mTOR inhibitor |
| Bosutinib | 57.65 ± | 10.30 | 35.82 ± | 5.54 | 36.20 ± | 7.65 | 0.026 | 0.030 | tyrosine kinase |
| | | | | | | | | | inhibitor (BCR- |
| | | | | | | | | | Abl, Src family |
| | | | | | | | | | kinase) |
| Belinostat | 54.56 ± | 4.92 | 79.38 ± | 3.41 | 37.29 ± | 10.31 | 0.001 | 0.055 | histone |
| | | | | | | | | | deacetylase |
| | | | | | | | | | inhibitor |
| Sirolimus | 53.86 ± | 2.78 | 43.15 ± | 2.85 | 30.27 ± | 4.88 | 0.003 | 0.001 | mTOR inhibitor |
| Sunitinib | 53.00 ± | 8.71 | 34.85 ± | 8.76 | 38.55 ± | 11.15 | 0.044 | 0.130 | multi-targeted |
| | | | | | | | | | RTK inhibitor |
| | | | | | | | | | (VEGFR, PDGFR) |
| Gemcitabine | 52.68 ± | 2.66 | 50.73 ± | 8.68 | 37.43 ± | 11.02 | 0.731 | 0.093 | DNA synthesis; |
| hydrochloride | | | | | | | | | |
| Everolimus | 50.48 ± | 4.75 | 39.76 ± | 3.54 | 28.02 ± | 3.93 | 0.038 | 0.002 | mTOR inhibitor |
| Sorafenib | 49.65 ± | 15.29 | 13.24 ± | 7.39 | 8.77 ± | 8.18 | 0.020 | 0.013 | multi-targeted |
| | | | | | | | | | RTK inhibitor |
| | | | | | | | | | (VEGFR and |
| | | | | | | | | | PDGFR) |
| Bleomycin | 49.04 ± | 3.45 | 50.88 ± | 6.71 | 29.04 ± | 2.53 | 0.693 | 0.000 | antibiotics |
| sulfate | | | | | | | | | |
| Thioguanine | 47.63 ± | 5.54 | 13.78 ± | 5.10 | 30.81 ± | 5.81 | 0.000 | 0.011 | antimetabolites |
| Valrubicin | 45.35 ± | 21.71 | −4.92 ± | 7.16 | 2.82 ± | 4.06 | 0.022 | 0.040 | antibiotic |
| Pazopanib | 45.34 ± | 21.95 | 4.01 ± | 24.21 | 31.78 ± | 22.26 | 0.071 | 0.481 | multi-targeted |
| hydrochloride | | | | | | | | | RTK inhibitor |
| | | | | | | | | | (VEGFR, PDGFR, |
| | | | | | | | | | FGFR, c-Kit and |
| | | | | | | | | | c-Fms) |
| Clofarabine | 44.23 ± | 1.93 | 55.78 ± | 7.52 | 35.23 ± | 11.44 | 0.073 | 0.267 | antimetabolites |
| Paclitaxel | 42.90 ± | 4.11 | 47.99 ± | 3.25 | 41.64 ± | 4.36 | 0.146 | 0.727 | microtubule |
| | | | | | | | | | inhibitor |
| Vorinostat | 42.80 ± | 2.50 | 72.11 ± | 6.08 | 30.92 ± | 14.92 | 0.002 | 0.263 | Histone |
| | | | | | | | | | deacetylase |
| | | | | | | | | | inhibitors |
| Azacitidine | 42.64 ± | 4.84 | 44.27 ± | 6.66 | 31.51 ± | 3.90 | 0.744 | 0.022 | chemotherapeutic |
| | | | | | | | | | agents |
| Afatinib | 41.38 ± | 31.06 | 20.01 ± | 5.26 | 13.07 ± | 2.30 | 0.321 | 0.212 | multi-targeted |
| | | | | | | | | | RTK inhibitor |
| | | | | | | | | | (EGFR, HER2) |
| Dabrafenib- | 40.95 ± | 5.81 | 22.13 ± | 9.95 | −6.26 ± | 4.13 | 0.038 | 0.000 | BRAF inhibitor |
| mesylate | | | | | | | | | |
| Triethylene- | 40.60 ± | 5.76 | 46.10 ± | 7.11 | 20.73 ± | 6.02 | 0.341 | 0.006 | chemotherapeutic |
| melamine | | | | | | | | | agent |
| Cladribine | 40.23 ± | 3.53 | 57.01 ± | 6.49 | 33.35 ± | 11.53 | 0.013 | 0.386 | antimetabolites |
| Trametinib | 40.22 ± | 4.19 | 34.72 ± | 1.51 | −0.64 ± | 5.09 | 0.104 | 0.000 | MEK inhibitor |
| Oxaliplatin | 39.55 ± | 2.37 | 25.39 ± | 2.42 | 13.52 ± | 5.20 | 0.000 | 0.001 | chemotherapeutic |
| | | | | | | | | | agent |
| Vinblastine | 35.39 ± | 1.69 | 53.78 ± | 2.67 | 43.02 ± | 4.33 | 0.000 | 0.048 | microtubule |
| sulfate | | | | | | | | | inhibitor |
| Regorafenib | 35.36 ± | 3.79 | 56.71 ± | 4.94 | 34.68 ± | 9.16 | 0.001 | 0.910 | multi-targeted |
| | | | | | | | | | RTK inhibitor |
| | | | | | | | | | (VEGFR2, TIE2) |
| Vinorelbine | 35.26 ± | 3.43 | 51.59 ± | 2.84 | 36.04 ± | 5.83 | 0.001 | 0.850 | microtubule |
| tartrate | | | | | | | | | inhibitor |
| Etoposide | 34.62 ± | 3.82 | 47.96 ± | 5.14 | 26.52 ± | 7.47 | 0.013 | 0.162 | topoisomcrase |
| | | | | | | | | | inhibitor |
| Pralatrexate | 34.60 ± | 34.53 | −2.94 ± | 0.94 | −13.51 ± | 5.14 | 0.160 | 0.095 | folate analogue |
| | | | | | | | | | metabolic |
| | | | | | | | | | inhibitor |
| Vincristine | 32.81 ± | 4.16 | 49.75 ± | 2.60 | 29.41 ± | 9.93 | 0.002 | 0.613 | microtubule |
| sulfate | | | | | | | | | inhibitor |
| Fluorouracil | 32.67 ± | 3.70 | 20.48 ± | 6.08 | 16.43 ± | 3.45 | 0.032 | 0.001 | antimetabolites |
| Cytarabine | 30.30 ± | 4.96 | 42.88 ± | 12.84 | 27.99 ± | 9.66 | 0.191 | 0.728 | chemotherapeutic |
| hxdrochloride | | | | | | | | | agents |
| Romidepsin | 29.66 ± | 3.66 | 50.89 ± | 2.44 | 39.07 ± | 7.70 | 0.000 | 0.124 | Histone |
| | | | | | | | | | deacetylase |
| | | | | | | | | | inhibitors |
| Axitinib | 28.13 ± | 12.90 | 18.51 ± | 6.09 | 2.30 ± | 11.05 | 0.304 | 0.040 | multi-targeted |
| | | | | | | | | | RTK inhibitor |
| | | | | | | | | | (VEGFR, c-KIT, |
| | | | | | | | | | PDGFR) |
| Gefitinib | 26.69 ± | 5.05 | 11.93 ± | 4.12 | 19.20 ± | 19.54 | 0.008 | 0.561 | EGFR |
| | | | | | | | | | inhibitor |
| Ibrutinib | 24.52 ± | 8.83 | 31.64 ± | 16.99 | 25.81 ± | 10.98 | 0.551 | 0.880 | Bruton's tyrosine |
| | | | | | | | | | kinase |
| Idelalisib | 23.39 ± | 3.19 | 23.75 ± | 5.89 | 36.19 ± | 2.62 | 0.929 | 0.002 | phosphoinositide |
| | | | | | | | | | 3-kinase inhibitor |
| Irinotecan | 22.26 ± | 3.10 | 44.02 ± | 8.12 | 19.37 ± | 2.33 | 0.013 | 0.247 | topoisomerase |
| hydrochloride | | | | | | | | | inhibitor |
| Mechlore- | 21.76 ± | 2.99 | 41.12 ± | 8.77 | 14.22 ± | 6.46 | 0.026 | 0.137 | chemotherapeutic |
| thamine | | | | | | | | | agents |
| hydrochloride | | | | | | | | | |
| Cabazitaxel | 21.15 ± | 3.98 | 40.06 ± | 6.34 | 32.37 ± | 5.79 | 0.007 | 0.037 | microtubule |
| | | | | | | | | | inhibitor |
| Floxuridine | 19.64 ± | 3.56 | 23.25 ± | 2.06 | 10.72 ± | 2.36 | 0.192 | 0.014 | antimetabolites |
| Docetaxel | 18.42 ± | 7.10 | 38.82 ± | 6.01 | 31.54 ± | 3.95 | 0.009 | 0.041 | microtubule |
| | | | | | | | | | inhibitor |
| Uracil | 17.47 ± | 3.41 | 18.80 ± | 8.40 | 6.77 ± | 3.14 | 0.811 | 0.007 | chemotherapeutic |
| mustard | | | | | | | | | agents |
| Nilotinib | 13.11 ± | 4.53 | −7.48 ± | 8.48 | −9.36 ± | 7.28 | 0.028 | 0.016 | tyrosine kinase |
| | | | | | | | | | inhibitor (BCR- |
| | | | | | | | | | Abl, c-KIT, Lck) |
| Lomustine | 12.00 ± | 2.39 | 12.58 ± | 5.16 | 11.37 ± | 5.21 | 0.869 | 0.856 | chemotherapeutic |
| | | | | | | | | | agents |
| Olaparib | 11.51 ± | 2.51 | 16.80 ± | 7.5 | 10.97 ± | 2.08 | 0.317 | 0.782 | chemotherapeutic |
| | | | | | | | | | agent |
| Mercapto- | 10.90 ± | 6.64 | 6.10 ± | 5.02 | 0.45 ± | 1.76 | 0.360 | 0.068 | immunosuppressive |
| purine | | | | | | | | | drug |
| Chlorambucil | 10.85 ± | 4.46 | 22.00 ± | 1.90 | 3.69 ± | 3.26 | 0.016 | 0.070 | chemotherapeutic |
| | | | | | | | | | agents |
| Dexrazoxane | 10.82 ± | 4.08 | 13.37 ± | 6.52 | 5.76 ± | 6.04 | 0.589 | 0.281 | cardioprotective |
| | | | | | | | | | agent |
| Erlotinib | 10.67 ± | 13.50 | 35.28 ± | 16.84 | 25.46 ± | 13.32 | 0.098 | 0.226 | antimetabolites |
| hydrochloride | | | | | | | | | |
| Tamoxifen | 10.45 ± | 5.96 | 24.32 ± | 19.11 | 37.26 ± | 11.75 | 0.304 | 0.020 | selective |
| citrate | | | | | | | | | estrogen-receptor |
| | | | | | | | | | modulator |
| Thiotepa | 10.25 ± | 7.23 | 21.58 ± | 4.76 | 8.06 ± | 1.92 | 0.071 | 0.642 | chemotherapeutic |
| | | | | | | | | | agents |
| Pipobroman | 10.05 ± | 1.23 | 15.75 ± | 6.46 | 3.55 ± | 4.34 | 0.225 | 0.076 | chemotherapeutic |
| | | | | | | | | | agents/alkylating |
| | | | | | | | | | agent |
| Melphalan | 9.53 ± | 3.93 | 11.09 ± | 5.24 | −1.25 ± | 2.16 | 0.694 | 0.010 | alkylating agents |
| hxdrochloride | | | | | | | | | |
| Decitabine | 8.76 ± | 2.58 | 3.11 ± | 3.23 | −1.12 ± | 5.45 | 0.058 | 0.043 | DNA |
| | | | | | | | | | methyltransferase |
| | | | | | | | | | inhibitor |
| Raloxifene | 7.46 ± | 1.71 | 9.41 ± | 7.33 | 28.24 ± | 26.13 | 0.681 | 0.262 | selective estrogen |
| | | | | | | | | | receptor |
| | | | | | | | | | modulator |
| | | | | | | | | | (SERM) |
| Carmustine | 7.11 ± | 6.51 | 11.20 ± | 3.21 | 3.32 ± | 2.51 | 0.380 | 0.401 | chemotherapeutic |
| | | | | | | | | | agents |
| Exemestane | 6.56 ± | 2.99 | 5.08 ± | 3.26 | −4.29 ± | 2.60 | 0.585 | 0.003 | aromatase |
| | | | | | | | | | inhibitors |
| Lapatinib | 6.44 ± | 5.74 | −2.48 ± | 5.78 | −3.81 ± | 7.41 | 0.107 | 0.110 | multi-targeted |
| | | | | | | | | | RTK inhibitor |
| | | | | | | | | | (HER2, EGFR) |
| Letrozole | 6.07 ± | 3.14 | 4.31 ± | 6.48 | −0.85 ± | 5.95 | 0.693 | 0.141 | Aromatase |
| | | | | | | | | | inhibitors |
| Dasatinib | 6.05 ± | 14.11 | 18.57 ± | 13.42 | 16.99 ± | 9.69 | 0.308 | 0.316 | multi-BCR/Abl |
| | | | | | | | | | and Src family |
| | | | | | | | | | tyrosine kinase |
| | | | | | | | | | inhibitor |
| Procarbazine | 6.02 ± | 7.28 | 0.84 ± | 5.66 | −3.53 ± | 1.96 | 0.370 | 0.105 | chemotherapeutic |
| hxdrochloride | | | | | | | | | agents |
| Plerixafor | 5.99 ± | 3.13 | 3.15 ± | 4.91 | −2.43 ± | 2.55 | 0.436 | 0.012 | immunostimulant |
| Celecoxib | 5.55 ± | 4.73 | 0.37 ± | 5.65 | −10.16 ± | 5.65 | 0.270 | 0.011 | COX-2 selective |
| | | | | | | | | | nonsteroidal |
| | | | | | | | | | anti-inflammatory |
| | | | | | | | | | drug |
| Abiraterone | 5.33 ± | 9.30 | −6.50 ± | 4.67 | −7.15 ± | 1.25 | 0.114 | 0.102 | antiandrogen |
| | | | | | | | | | activity |
| Pemetrexed | 5.13 ± | 3.08 | 2.48 ± | 4.32 | −2.57 ± | 4.77 | 0.424 | 0.064 | chemotherapy |
| disodium salt | | | | | | | | | drugs called |
| | | | | | | | | | folate |
| | | | | | | | | | antimetabolites |
| Mitotane | 4.55 ± | 2.29 | 1.39 ± | 4.88 | −0.12 ± | 2.25 | 0.365 | 0.045 | antimetabolites |
| Ifosfamide | 4.47 ± | 5.64 | −3.81 ± | 1.30 | −0.66 ± | 3.83 | 0.081 | 0.247 | chemotherapeutic |
| | | | | | | | | | agents |
| Lenalidomide | 4.46 ± | 2.50 | 1.27 ± | 8.73 | −2.09 ± | 4.04 | 0.580 | 0.063 | anti-tumor effect, |
| | | | | | | | | | inhibition of |
| | | | | | | | | | angiogenesis, and |
| | | | | | | | | | immunomodulatory |
| | | | | | | | | | role |
| Anastrozole | 3.95 ± | 4.02 | 4.23 ± | 8.05 | −1.07 ± | 4.15 | 0.960 | 0.183 | aromatase |
| | | | | | | | | | inhibitors |
| Fulvestrant | 3.89 ± | 4.72 | −4.95 ± | 2.87 | −2.52 ± | 8.38 | 0.040 | 0.304 | estrogen receptor |
| | | | | | | | | | antagonist |
| Idarubicin | 3.36 ± | 2.99 | 1.79 ± | 6.96 | −4.82 ± | 3.36 | 0.737 | 0.020 | antitumor |
| hydrochloride | | | | | | | | | antibiotics. |
| Nelarabine | 3.00 ± | 3.31 | 4.75 ± | 5.71 | −0.28 ± | 1.34 | 0.667 | 0.188 | chemotherapeutic |
| | | | | | | | | | agents |
| Megestrol- | 2.95 ± | 4.32 | 1.49 ± | 4.47 | −7.37 ± | 3.75 | 0.700 | 0.021 | potent agonist of |
| acetate | | | | | | | | | the progesterone |
| | | | | | | | | | receptor |
| Cyclophos- | 2.88 ± | 5.30 | −1.06 ± | 4.30 | −4.00 ± | 3.45 | 0.357 | 0.117 | chemotherapeutic |
| phamide | | | | | | | | | agents |
| Imiquimod | 2.68 ± | 3.32 | −0.75 ± | 3.89 | −3.32 ± | 4.11 | 0.290 | 0.099 | immune response |
| | | | | | | | | | modifier (IRM) |
| Benda- | 2.12 ± | 3.14 | 3.19 ± | 5.22 | −0.61 ± | 2.47 | 0.774 | 0.284 | chemotherapeutic |
| mustine | | | | | | | | | agents, alkylating |
| hydrochloride | | | | | | | | | agent |
| Imatinib | 1.90 ± | 4.30 | 12.44 ± | 23.61 | −5.42 ± | 4.44 | 0.499 | 0.086 | multi-targeted |
| | | | | | | | | | RTK inhibitor |
| | | | | | | | | | (BCR-Abl, |
| | | | | | | | | | c-KIT, PDGFR) |
| Vismodegib | 1.57 ± | 4.46 | −1.48 ± | 5.81 | −9.97 ± | 2.25 | 0.499 | 0.013 | antagonist of the |
| | | | | | | | | | smoothened |
| | | | | | | | | | receptor (SMO) |
| Carboplatin | 1.26 ± | 2.89 | 3.77 ± | 1.74 | −3.94 ± | 2.60 | 0.255 | 0.060 | chemotherapeutic |
| | | | | | | | | | agent |
| Fludarabine | 1.21 ± | 1.08 | 0.14 ± | 5.94 | −2.30 ± | 3.90 | 0.778 | 0.218 | chemotherapeutic |
| phosphate | | | | | | | | | agents |
| Cisplatin | 1.15 ± | 2.87 | −4.42 ± | 4.54 | −6.87 ± | 5.51 | 0.278 | 0.158 | chemotherapeutic |
| | | | | | | | | | agents |
| Capecitabine | 1.06 ± | 7.33 | 0.47 ± | 5.83 | −1.49 ± | 3.55 | 0.876 | 0.326 | chemotherapeutic |
| | | | | | | | | | agents |
| Vemurafenib | 0.70 ± | 3.13 | −8.56 ± | 2.96 | −4.40 ± | 5.67 | 0.060 | 0.230 | B-Raf inhibitor |
| Amifostine | 0.66 ± | 4.19 | −8.91 ± | 7.17 | −11.51 ± | 3.99 | 0.157 | 0.057 | cytoprotective |
| | | | | | | | | | adjuvant |
| Busulfan | 0.26 ± | 1.91 | 1.68 ± | 2.47 | 0.46 ± | 3.24 | 0.560 | 0.940 | chemotherapeutic |
| | | | | | | | | | agents |
| Tretinoin | 0.11 ± | 4.76 | −3.57 ± | 2.74 | −7.14 ± | 6.55 | 0.257 | 0.166 | retinoic acid |
| | | | | | | | | | receptors agonist |
| Methoxsalen | −0.04 ± | 1.91 | −1.93 ± | 2.06 | −1.69 ± | 3.24 | 0.289 | 0.482 | furanocoumarins |
| Allopurinol | −0.18 ± | 4.76 | 1.79 ± | 4.53 | −1.38 ± | 2.80 | 0.621 | 0.723 | antimetabolites |
| Arsenic | −0.26 ± | 2.86 | 1.49 ± | 3.99 | 2.81 ± | 4.60 | 0.562 | 0.371 | unclear |
| trioxide | | | | | | | | | |
| Zoledronic | −0.37 ± | 4.93 | −8.23 ± | 9.55 | −11.71 ± | 5.29 | 0.267 | 0.035 | anti-bone- |
| acid | | | | | | | | | resorption |
| Streptozocin | −0.95 ± | 6.61 | −5.23 ± | 5.62 | −5.84 ± | 6.97 | 0.427 | 0.412 | chemotherapeutic |
| | | | | | | | | | agents |
| Temozolo- | −1.03 ± | 2.84 | 3.01 ± | 3.34 | 0.12 ± | 3.03 | 0.162 | 0.649 | chemotherapeutic |
| mide | | | | | | | | | agents |
| Amino- | −1.31 ± | 5.00 | 3.76 ± | 6.35 | −3.60 ± | 4.41 | 0.321 | 0.575 | porphyrin |
| levulinic acid | | | | | | | | | synthesis |
| hydrochloride | | | | | | | | | inhibitor |
| Pomalido- | −1.48 ± | 5.61 | 5.37 ± | 5.50 | −4.53 ± | 2.86 | 0.182 | 0.445 | inhibition of |
| mide | | | | | | | | | angiogenesis, and |
| | | | | | | | | | immunomodulatory |
| | | | | | | | | | role |
| Thalidomide | −2.35 ± | 5.55 | −1.60 ± | 8.12 | −5.38 ± | 2.54 | 0.900 | 0.437 | immunomodulatory |
| | | | | | | | | | drug |
| Estramustine | −2.46 ± | 3.51 | −4.58 ± | 1.90 | −3.54 ± | 11.35 | 0.403 | 0.883 | antimicrotubule |
| phosphate | | | | | | | | | |
| sodium | | | | | | | | | |
| Pentostatin | −2.59 ± | 3.70 | −3.07 ± | 3.80 | −5.53 ± | 4.48 | 0.880 | 0.416 | chemotherapeutic |
| | | | | | | | | | agents |
| Hydroxyurea | −2.83 ± | 3.89 | −2.21 ± | 6.97 | −1.53 ± | 1.92 | 0.898 | 0.630 | antimetabolites |
| Methotrexate | −2.85 ± | 13.31 | −3.40 ± | 8.89 | −7.58 ± | 6.88 | 0.955 | 0.610 | antimetabolites |
| Altretamine | −3.57 ± | 4.87 | 2.67 ± | 4.32 | −0.88 ± | 4.13 | 0.120 | 0.493 | chemotherapeutic |
| | | | | | | | | | agents |
| Dacarbazine | −3.94 ± | 2.38 | 1.88 ± | 5.20 | −3.57 ± | 1.78 | 0.150 | 0.839 | alkylating agents |

TABLE 2
List of candidate drugs and IC50 Values

| | IC50 (μM) | | | |
|---|---|---|---|---|
| Compounds | L914F | WT | HUVEC | Targets |
| Sirolimus | <0.03 | <0.03 | <0.03 | mTOR inhibitor |
| Temsirolimus | <0.03 | <0.03 | <0.03 | |
| Everolimus | <0.03 | <0.03 | <0.03 | |
| Ponatinib | 0.42 ± 0.03 | 0.89 ± 0.24 | 1.26 ± 0.42 | BCR-Abl inhibitor |
| Bosutinib | 3.54 ± 1.14 | 4.1 ± 0.68 | 5.16 ± 1.85 | |
| Nilotinib | 3.61 ± 0.03 | 4.98 ± 0.90 | 6.39 ± 1.18 | |
| Cabozantinib | 3.06 ± 0.27 | 2.89 ± 1.16 | 1.35 ± 0.20 | multi-targeted RTK |
| | | | | inhibitor (c-Met, |
| | | | | VEGFR) |
| Vandetanib | >10 | 5.63 ± 0.24 | 6.20 ± 0.95 | multi-targeted RTK |
| | | | | inhibitor (VEGFR, |
| | | | | EGFR, RET) |
| Sorafenib | >10 | >10 | 6.84 ± 2.23 | multi-targeted RTK |
| | | | | inhibitor (VEGFR, |
| | | | | PDGFR) |
| Ceritinib | 9.03 ± 1.38 | 8.66 ± 2.98 | 8.13 ± 2.65 | ALK inhibitor |
| Dabrafenib | >10 | >10 | >10 | BRAF inhibitor |
| mesylate | | | | |

TABLE 3
Combination index values. This table includes Combination Index (CI) value of combination test including three
ABL kinase inhibitors with rapamycin.

| | Ponatinib | | | Nilotinib | | | Bosutinib | | |
|---|---|---|---|---|---|---|---|---|---|
| Rapamycin | L914F | WT | HUVEC | L914F | WT | HUVEC | L914F | WT | HUVEC |
| Combination | 0.67 ± 0.26 | 0.74 ± 0.32 | 0.89 ± 0.24 | 0.8 ± 0.28 | 0.58 ± 0.15 | 0.71 ± 0.27 | 1.49 ± 0.31 | 1.72 ± 0.41 | 1.47 ± 0.41 |
| Index | | | | | | | | | |

According to the International Society for the Study of Vascular Anomalies (ISSVA), VMs are subdivided into sporadic VM, inherited cutaneomucosal venous malformation (VMCM), blue rubber bleb nevus syndrome (BRBNS) and glomuvenous malformations (GVM)(9). About 30-40% of sporadic VMs are associated with mutations in the endothelial cell tyrosine kinase receptor TIE2 gene (TEK)(10). Most TIE2 mutations are localized in the intracellular, tyrosine kinase (TK) or insert kinase domain, and the most common substitution is L914F (11). When TIE2 mutations, formerly identified in patients, are expressed in human umbilical vein endothelial cells (HUVEC), they induce ligand-independent phosphorylation of TIE2 in variable degree (11). Activated TIE2 increases the phosphorylation of downstream pathways such as PI3K/AKT and MAPK to promote EC survival (12). Applicant previously showed that the TIE2-activating mutation L914F is sufficient to induce HUVECs to form VM lesions in nude mice. These murine VMs appear as bluish lesions that expand over time and have similar histological characteristics to patient VMs (13). Recently, somatic mutations in the catalytic subunit of class I phosphoinositide 3-kinases (PIK3CA) have been associated with about 20% of VM cases (10, 14, 15). PIK3CA mutations have been also reported in several types of cancer (16), overgrowth syndromes (17-19), and lymphatic malformations (20-22).

The mammalian target of rapamycin (mTOR), integrates signals from activation of the PI3K/AKT pathway to regulate multiple cellular processes including cell growth and proliferation (23). Enhanced mTOR signaling can increase expression of vascular endothelial growth factor (VEGF) thus promoting pathological angiogenesis (23). The mTOR inhibitor rapamycin showed suppression of the TIE2-L914F-induced AKT phosphorylation, and its effect was more potent than direct TIE2 kinase inhibition. Furthermore, rapamycin inhibited murine VM lesion expansion but failed to promote regression (13). Clinical trials of rapamycin in difficult-to-treat VM patients and complicated vascular anomalies have shown improved clinical symptoms and tolerated toxicity (6, 7). Rapamycin has become a new therapeutic option for VM patients who are refractory to standard care, but its moderate effectiveness to induce significant lesion regression could limit its further use (7, 13).

The Abelson (ABL) family of non-receptor tyrosine kinases (ABL1 and ABL2) have critical roles in regulating cytoskeletal reorganization and cellular functions such as proliferation and survival (24). Enhanced ABL expression occurs as a consequence of chromosome translocation of BCR-ABL1 fusion proteins, which promotes constitutive ABL kinase activity and drive human leukemia. Imatinib was the first ABL kinase ATP-competitive inhibitor approved by Food and Drug Administration (FDA) for chronic myelogenous leukemia (CML) and Philadelphia-positive (Ph+) leukemia treatment. In the past two decades, the ATP-competitive ABL kinase inhibitors Ponatinib, Nilotinib and Bosutinib have been shown to improve efficacy and overcome Imatinib resistance. These small molecule inhibitors, which were initially developed to target BCR-ABL1 fusion protein in cancer, are also under investigation to treat diverse pathologies with hyper-active ABL kinases (24, 25).

Treplaceo explore efficacious and potent therapies for VM, Applicant performed an unbiased screening of FDA-approved drugs and shown that mTOR inhibitors and ABL kinase inhibitors were the most potent drugs effective in decreasing HUVEC-TIE2-L914F cell proliferation. Applicant found that c-ABL was constitutively phosphorylated downstream of the TIE2-L914F. Thereby, here Applicant tested the hypothesis that ABL kinase inhibitors when combined with rapamycin are efficient in inducing significant regression of VM lesions.

ABL Kinase Inhibitors Decrease HUVEC-TIE2-L914F Proliferation

Retrovirally-transfected TIE2-L914F HUVECs (HUVEC-TIE2-L914F) were proven to be a powerful tool for the pre-clinical testing of rapamycin as a candidate for the treatment of murine and human VM(13). To identify drugs able to induce VM regression, Applicant performed an unbiased screening of Food and Drug Administration (FDA)-oncology approved drugs and tested their ability to inhibit cell proliferation in HUVEC-TIE2-L914F. By comparing proliferation rates between HUVEC-TIE2-L914F and HUVEC-TIE2-wild type (WT) or non-transfected, Applicant identified 11 candidate drugs that were significantly more selective (P<0.05) for the HUVEC-TIE2-L914F than for the other two cell lines (Table S1). Applicant then calculated the half maximal inhibitory concentration (IC50) of these 11 drugs by 6 dilution doses from 10 μM to 30 nM (Table S2). The mTOR inhibitors tested (Sirolimus, Temsirolimus and Everolimus) showed dose-independent (30 nM-10 μM) inhibition curves in HUVEC-TIE2-L914F cells and IC50<30 nM, which is consistent with previous studies (26, 27). Interestingly, in HUVEC-TIE2-L914F, the IC50 of three ABL kinase inhibitors, Ponatinib, Bosutinib and Nilotinib were 0.42±0.03 μM, 3.54±1.14 μM and 3.61±0.03 μM respectively, while the IC50s were higher in the control cells HUVEC-TIE2-WT and HUVEC (Table S2). These results suggest that ABL kinase inhibitors could be selected as candidate drugs to test for murine VM treatment. It also indicates that ABL kinase may play an important role in VM expansion.

c-ABL is Constitutively Phosphorylated in HUVEC-TIE2-L914F

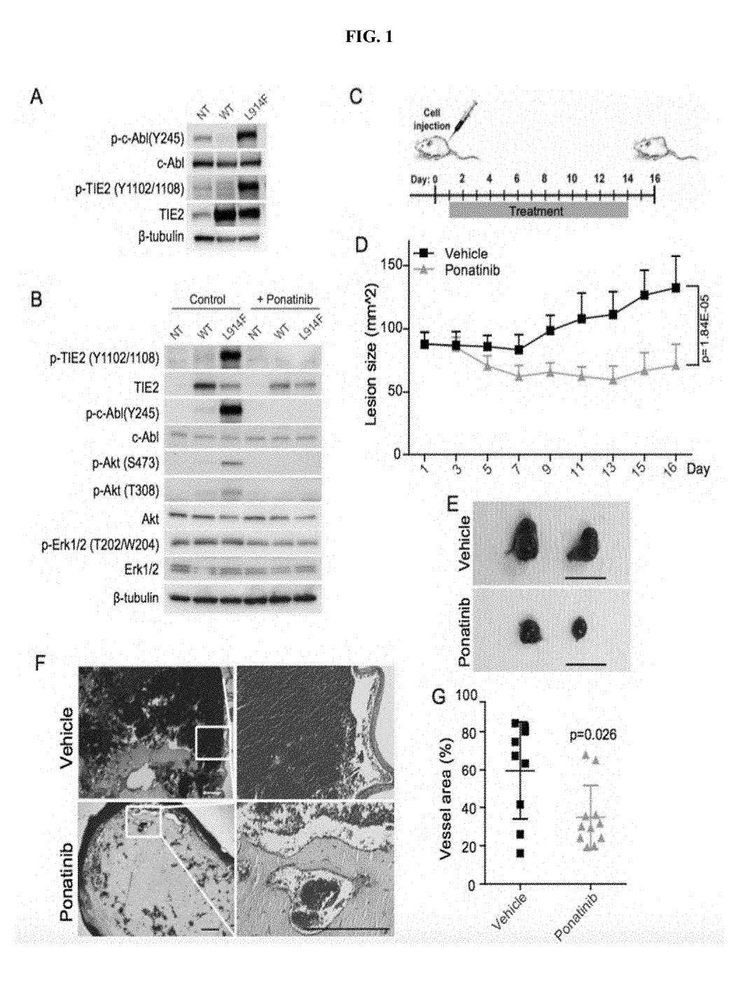

Applicant first assessed whether ABL kinase was highly-activated in the TIE2-mutated HUVECs as c-ABL is the common target of the three ABL kinase inhibitors (Ponatinib, Bosutinib and Nilotinib) that affected HUVEC-TIE2-L914F proliferation. Applicant's results revealed that phosphorylation of c-ABL was strongly increased in HUVEC-TIE2-L914F, compared to HUVEC-TIE2-WT and normal HUVEC (FIG. 1A). The three BCR-ABL inhibitors are ATP-competitive inhibitors that target multiple tyrosine kinases(24). Ponatinib targets both ABL kinase and TIE2 (24), and showed more efficacy than Bosutinib and Nilotinib (Table S2) in inducing cell proliferation inhibition in HUVEC-TIE2-L914F. Applicant tested for the effects of Ponatinib treatment on HUVEC-TIE2-L914F and detected a diminished phosphorylation of both TIE2 and c-ABL, and reduced activation of the TIE2 downstream effector AKT (FIG. 1B). These results suggest that the ABL kinase inhibitor Ponatinib inhibits the TIE2-L914F-induced activation of c-ABL and the downstream PI3K/AKT signaling.

Ponatinib Inhibits VM Formation In Vivo.

Applicant further assessed Ponatinib efficacy in the VM murine model Applicant previously established (13). In previous studies, Ponatinib at 30 mg/kg was shown to be effective in preventing BCR-ABL^{T315I}-induced tumor growth in murine xenografts (28). In Applicant's VM murine model, following HUVEC-TIE2-L914F cell injection, mice were treated for 14 days with Ponatinib (30 mg/kg) or vehicle (schematic in FIG. 1C). At the end of the treatment, lesion size was significantly smaller (P=1.84E-05) in the Ponatinib-treated group, when compared to the vehicle-treated group (FIGS. 1D and 1E). Hematoxylin and Eosin (H&E) staining revealed that Ponatinib treatment significantly (P=0.026) prevented formation of enlarged vessels (FIGS. 1F and 1G). Meanwhile, there was no effect on mouse body weight. These data suggest that Ponatinib inhibits lesion expansion and vessel formation in HUVEC-TIE2-L914F injected mice.

Combination of Ponatinib with Rapamycin Shows Synergetic Inhibition of Cell Proliferation in HUVEC-TIE2-L914F

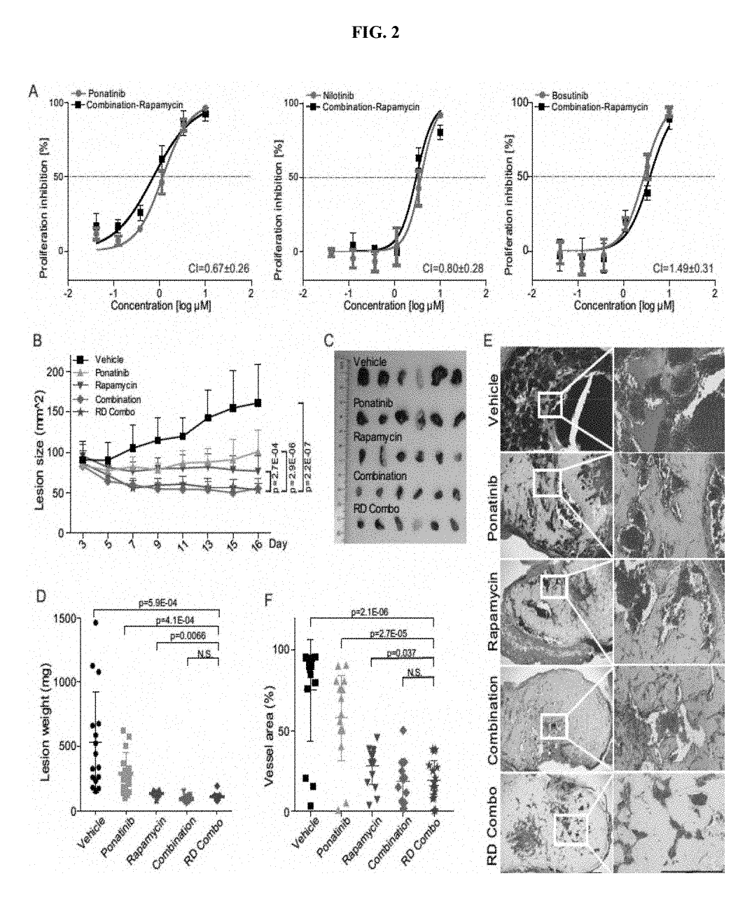

Due to mTOR inhibitor rapamycin showing modest lesion regression in VMs clinical trial(13), Applicant sought to determine whether combination of ABL kinase inhibitor/s with rapamycin was more potent than single drug treatment. To assess synergism between the two drugs, Applicant calculated the combination index (CI) value. In Applicant's study, rapamycin showed a dose-independent proliferation inhibition (Table S1) which is an ineligible prerequisite for the Chou-Talalay method, the gold standard for dual combination evaluation. Based on published studies, Applicant defined dryg synergism for a CI value ≦0.8 (a more stringent criteria than CI≦1 as proposed by the Chou-Talalay method) (26, 29). In the assays, the three ABL kinase inhibitors, Ponatinib, Bosutinib or Nilotinib were tested at 6 dose dilutions from 10 μM to 30 nM, while rapamycin was used at 10 nM, a concentration sufficient to inhibit AKT in the HUVEC-TIE2-L914F (13). Proliferation inhibition curves show that there was a significant shift of IC50 to a lower value in HUVEC-TIE2-L914F when rapamycin was combined with Ponatinib (FIG. 2A) and the mean CI value was 0.67±0.26, indicative of synergism, while for rapamycin with Nilotinib or with Bosutinib it was ≧0.8 (Table S3). These data suggest that combination of Ponatinib with rapamycin has a synergetic anti-proliferative effect on TIE-mutated HUVEC.

Combination of Ponatinib with Rapamycin Suppresses VM Lesion Expansion

Applicant next tested the efficacy and safety of this novel drug combination in the treatment of murine VM. First, Applicant analyzed the effects of the combination Ponatinib with rapamycin and compared to monotherapy; then, to identify the minimal effective dose of the combination, Applicant tested two reduced-dose regimens. Mice were injected with HUVEC-TIE2-L914F cells and subsequently given vehicle, Ponatinib (30 mg/kg), rapamycin (2 mg/kg), combination and reduced-dose combination (RD Combo, 20 mg/kg Ponatinib+1 mg/kg rapamycin). After 15 days of treatment, lesion size and weight in the combination group were smaller compared to monotherapy and vehicle treated groups. More interestingly, RD Combo treatment showed a similar reduction of lesion size and lesion weight (FIGS. 2B and 2C). The H&E staining of lesion explants showed that RD Combo prevented vessel enlargement and there was no difference between RD Combo and combination treatment (FIGS. 2E and 2F). The combination treatment induced a significant weight loss (P=0.04 and P=0.016) at days 9 and 11 which was not seen in the RD Combo group. These results suggest that: 1-drug combination is more effective than monotherapy; 2-reduced dose drug combination achieved similar effect on lesion expansion inhibition without affecting mouse weight.

Reduced Dose Drug Combination Induces VM Regression

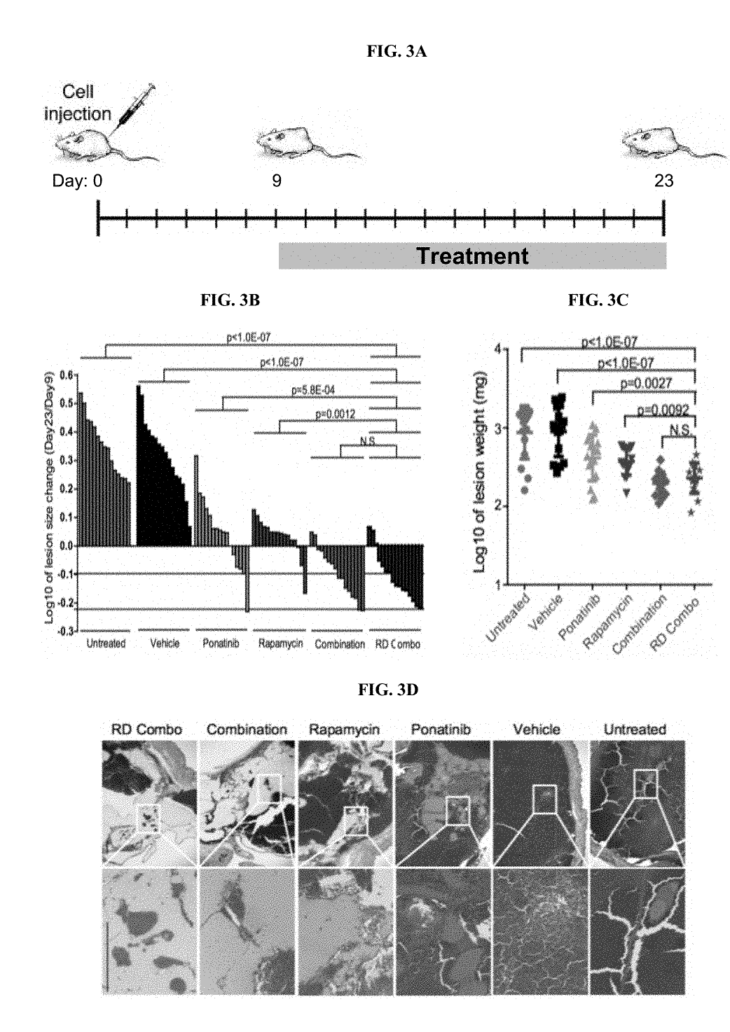

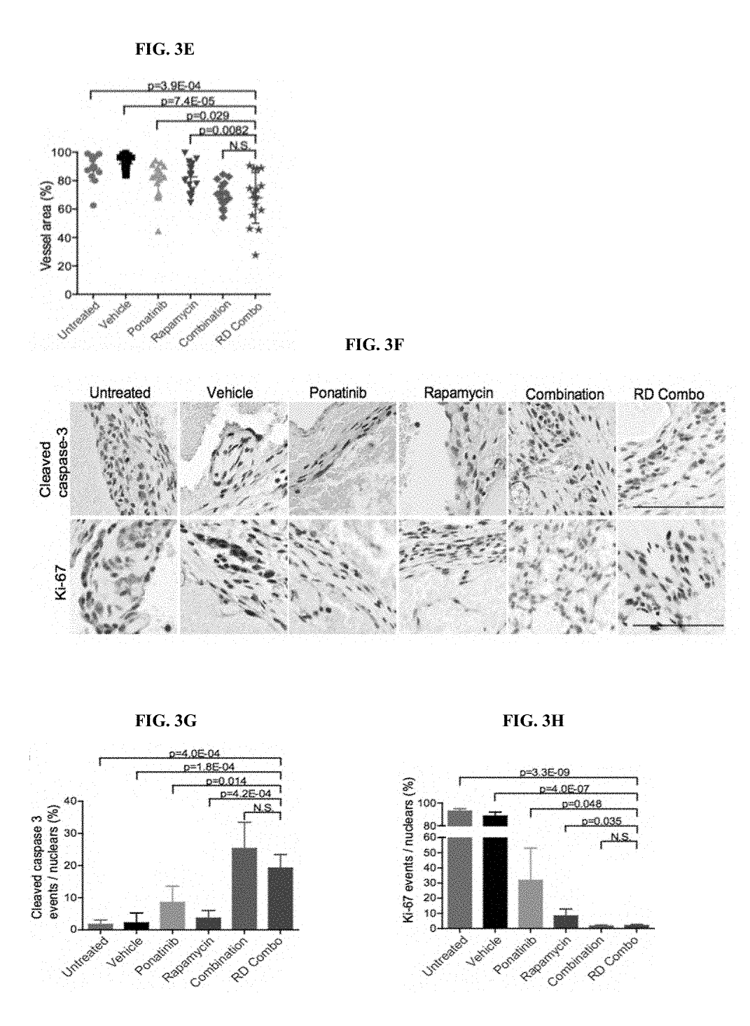

Rapamycin monotherapy has showed partial clinical response in VM patients but lesions failed to regress significantly in size (6, 7). To assess whether combining rapamycin with Ponatinib could induce a significant regression of pre-formed VM lesions, drug treatment started at day 9, when the average VM lesion size reached about 100 mm² (schematic in FIG. 3A). Combination and RD Combo treatment induced regression of most of the VM lesions (14/16 and 13/16 respectively), while only 5/16 and 2/16 lesions shrunk in the Ponatinib and rapamycin monotherapy group respectively (FIG. 3B). Weight of lesions in RD Combo were also smaller than monotherapy and vehicle groups (FIG. 3C). Analysis of lesion histology showed that combination and RD Combo treatments both inhibited vascular area expansion (FIG. 3D and FIG. 3E). No significant mouse weight loss was detected in response to drug combination at the concentrations Applicant tested. Furthermore, cleaved caspase-3 staining revealed that apoptotic cells were increased in number in in the RD combo (FIGS. 3F and 3G). Conversely, Ki-67 expressing proliferative cells were reduced in Ponatinib and rapamycin monotherapy, but barely detectable in the combination and RD combo treated lesions (FIGS. 3F and 3H). These results suggest that reduced-dose of the combination therapy Ponatinib with rapamycin induced significant murine VM lesion regression.

Drug Combination Show Minimal Side Effects Upon Long-Term Treatment

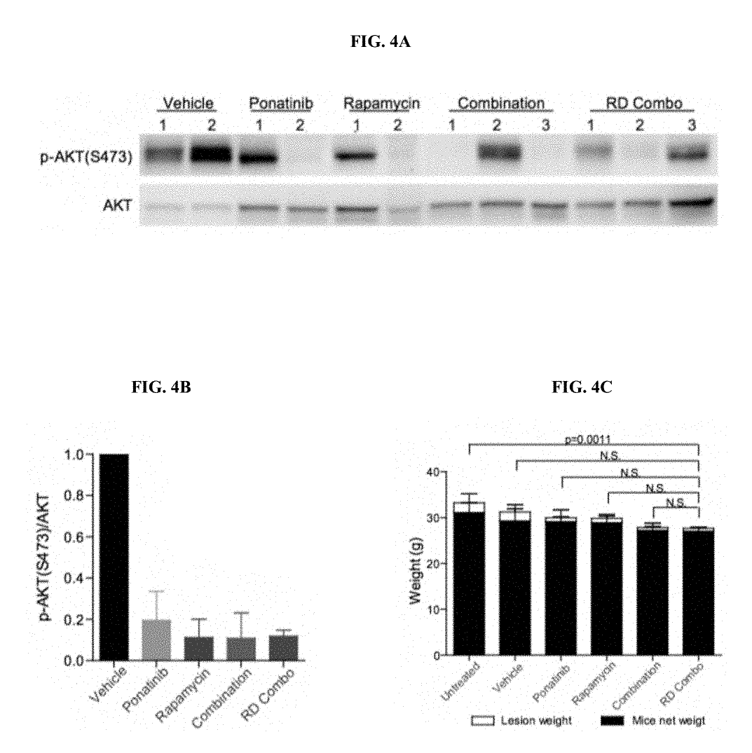

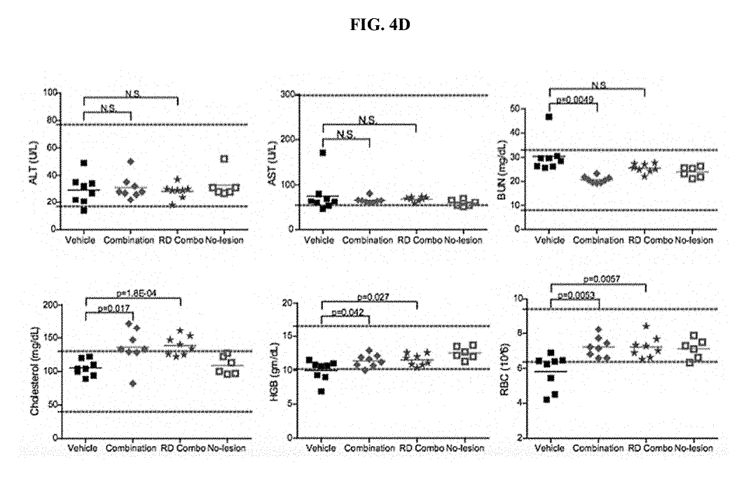

Applicant further assessed efficacy and safety of long-term drug combination treatment. Treatment started at day 12, when lesions reached 130 mm², and lasted 4 weeks. Combination and RD Combo treatment induced marked lesion regression. No lesion rebound was recorded. AKT phosphorylation at Ser473 in treated VM lesions were lower compared to vehicle group (FIGS. 4A and 4B). Mouse death was not recorded upon combination treatment, and net mouse weight in combination and RD combo group was similar to the vehicle treated (FIG. 4C). To analyze the drug combination toxicity, Applicant performed biochemical profiling and CBC (Complete Blood Count) blood tests after 4 weeks of treatment. Values of AST (Aspartate Aminotransferase), ALT (Alanine Aminotransferase) and BUN (Blood Urea Nitrogen) in combination and RD Combo were all in the reference range for normal liver and kidney function, while elevation of cholesterol was consistent with hypercholesterolemia found in the rapamycin clinical trial for lymphangioleyomyomatosis (LAM) patients (30). VM lesions in mice induce a significant decrease of hemoglobin and red blood cell levels (31). Drug combination treatment resulted in normalization of hemoglobin levels and erythrocyte number. These results show that that long-term drug combination treatment did not induce major side effects.

Drug Combination Enhances Cell Apoptosis in HUVEC-TIE2-L914F

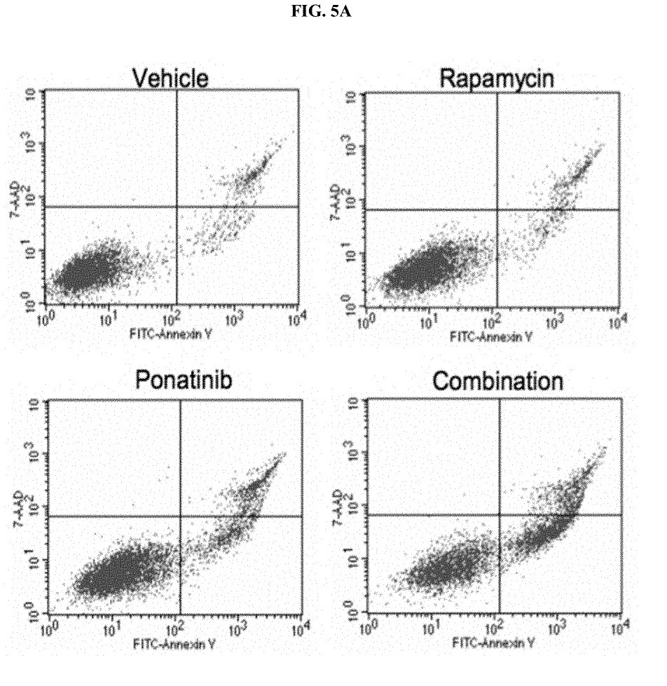

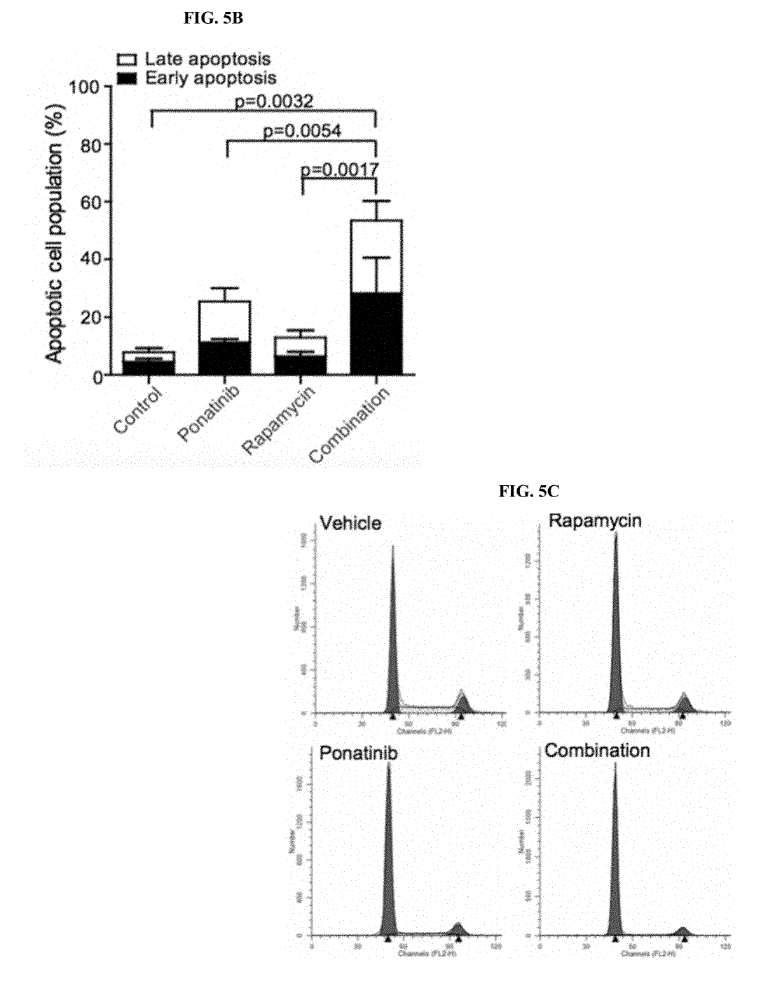

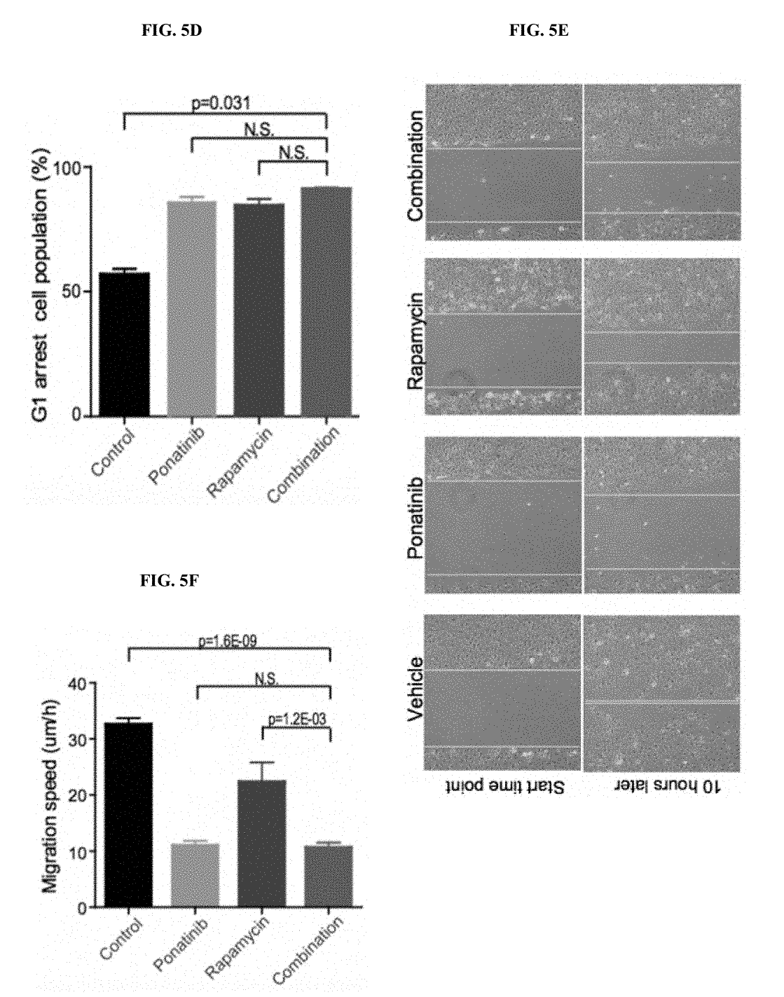

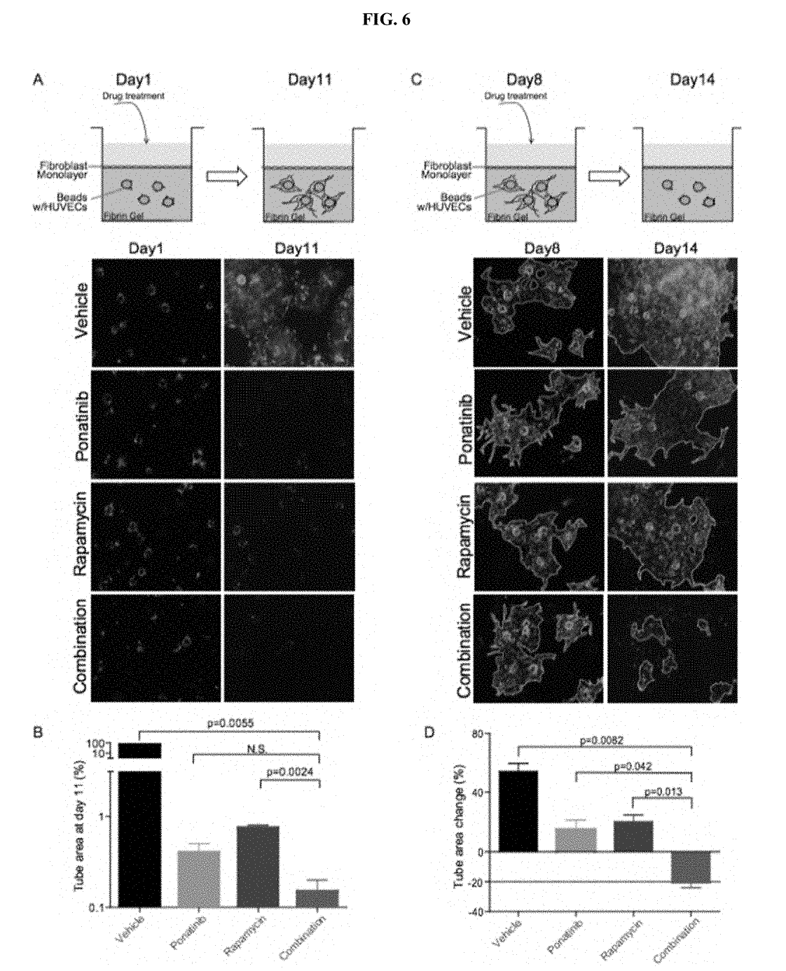

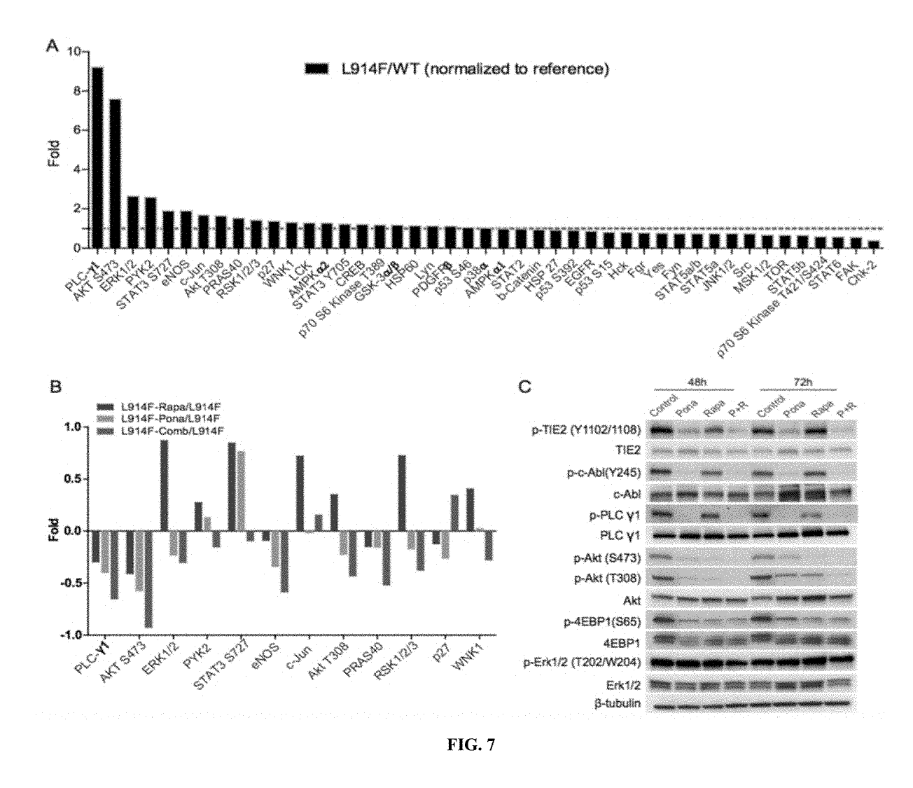

In order to study the cellular functions affected by the drug combination, Applicant assessed cell apoptosis in HUVEC-TIE2-L914F cells (FIG. 5A). Although rapamycin treatment alone promoted cell death in both Human Umbilical Artery Endothelial Cells (HUAECs) and normal HUVECs (32), in Applicant's experimental settings it did not induce significant cell apoptosis (13.00±5.02%) in TIE2-L914F-HUVECs compared to vehicle (DMSO) (7.89±1.71%) (FIGS. 5A and B). In contrast, Ponatinib treatment induced apoptosis (25.43±4.67%, P=0.023) and, when combined with rapamycin, this effect was greater than Ponatinib alone (53.47±5.02%, P=0.045 vs. Ponatinib alone). Furthermore, the expression of cleaved caspase-3 was higher in the drug combination treatment when compared to the DMSO control (P=1.6E-08) and Ponatinib alone (P=0.016).

Applicant next investigated the effects of drug combination on cell cycle arrest. Rapamycin was previously reported to block HUVEC cell cycle in the G1 phase (33). In line with these data, Applicant found that rapamycin treatment induced significant G0/G1 phase arrest (83.99±4.58%, P=0.0031) compared to DMSO control (56.29±4.06%, FIG. 5C and FIG. 5D). Similarly, Ponatinib treatment also induced HUVEC-TIE2-L914F to accumulate in G1 phase (85.41±3.57%, P=0.0015). Drug combination did not show any additional effect when compared to single drug treatments.

Applicant next established if the drug combination could affect migration. Here Applicant assessed the impact of combination treatment on HUVEC-TIE2-L914F migration by using a wound healing/scratch assay (FIG. 5E). Ponatinib and rapamycin treatment delayed cell migration speed compared to DMSO treatment, and in the combination treatment the migration speed was similar to the Ponatinib single drug-treatment (FIG. 5F). Taken together, these data indicate that, compared to single drug treatment, the combination of Ponatinib with rapamycin enhanced cell apoptosis but did not affect cell cycle and cell migration.

Drug Combination Promotes Regression of HUVEC-TIE2-L914F-Derived Vascular Channels

Three-dimensional (3D) systems such as the fibrin gel assay can recapitulate fundamental steps of the angiogenesis process in vitro(34). Compared to normal HUVEC, which formed thin tubes, HUVEC-TIE2-L914F formed large and expanding saccular channels akin to the VM vessels. Applicant evaluated the effect of the drug combination on HUVEC-TIE2-L914F-derived vascular channel/lumen formation and regression in this assay. To test the effect of the drug treatment on channel formation Applicant added rapamycin, Ponatinib or combination starting from day 1 (FIG. 6A), then imaged the same exact location on the well every other day. At day 11, compared to vehicle (DMSO) group where tube area increased about 75 folds, mono- and combination treatment almost completely inhibited tube formation (FIG. 6B). There was no statistical difference between mono- and combination treatments.

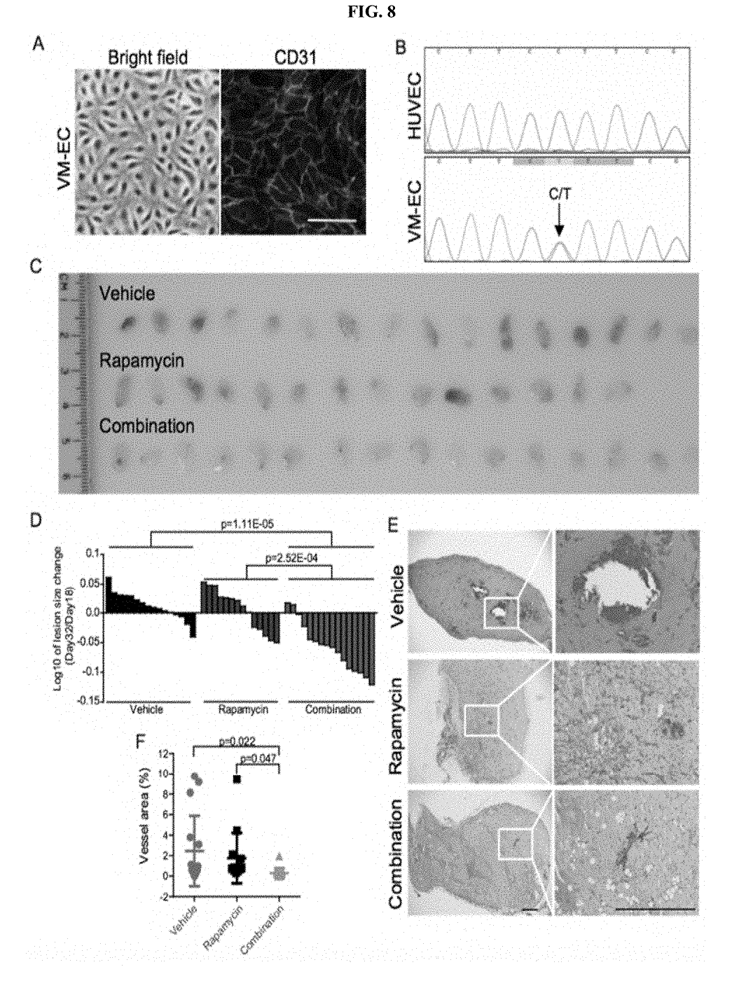

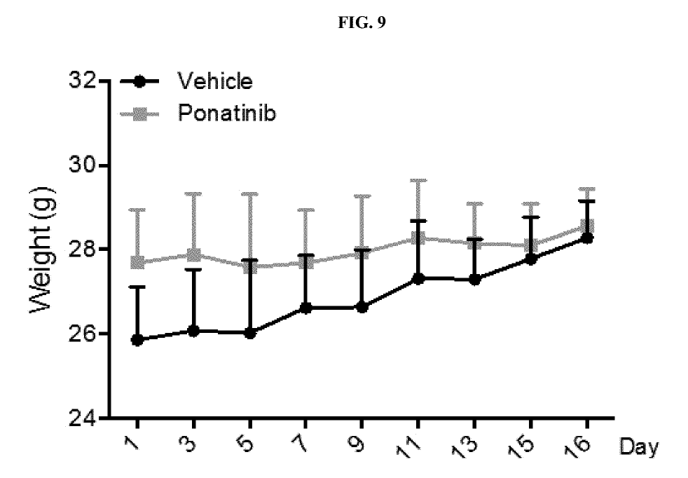

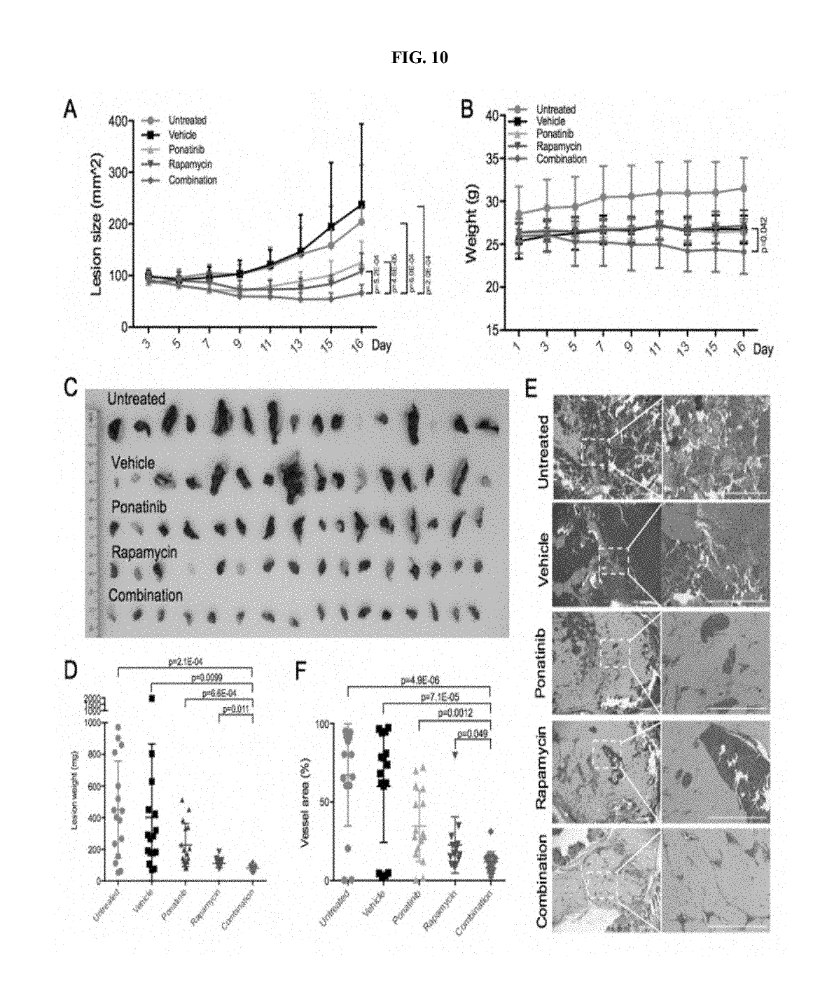

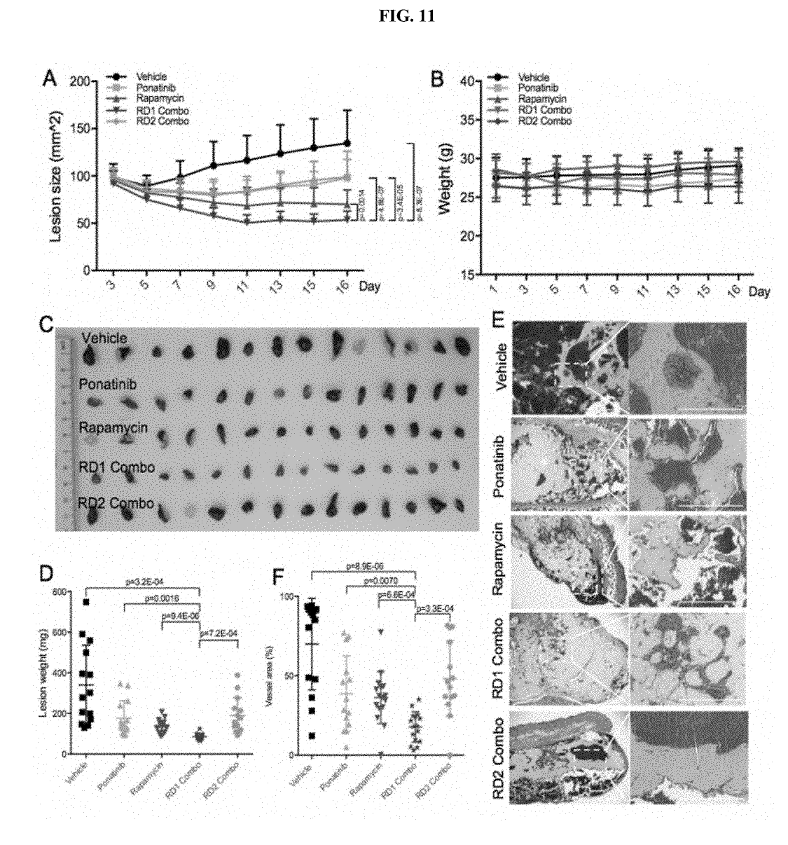

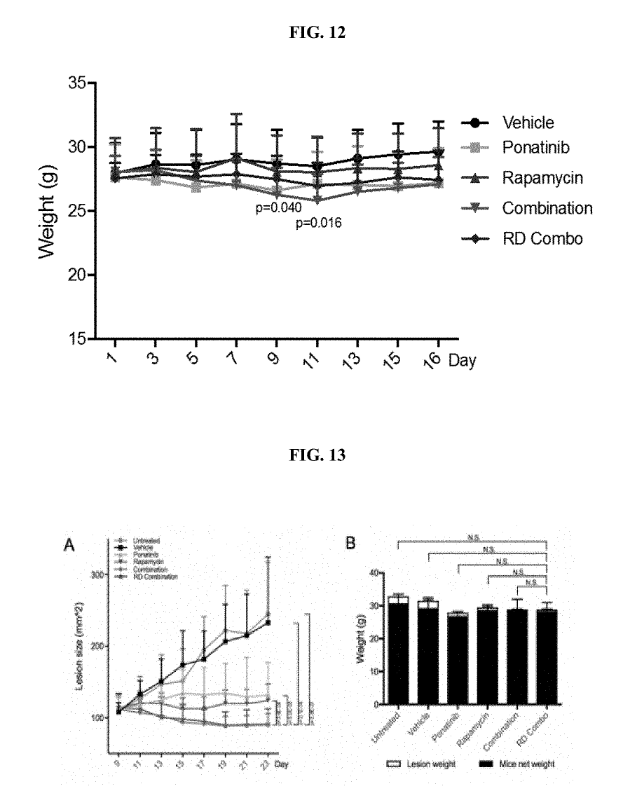

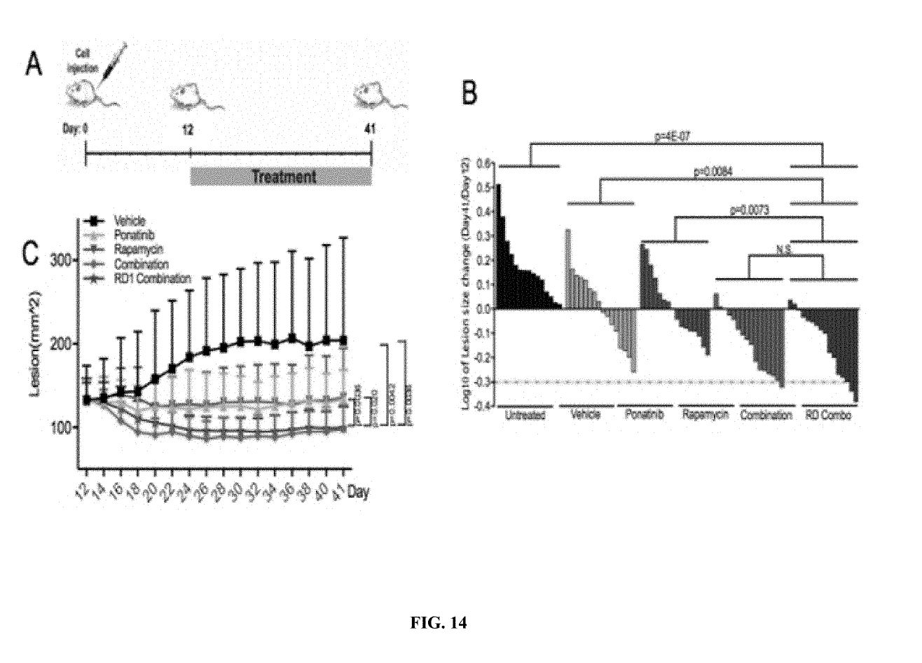

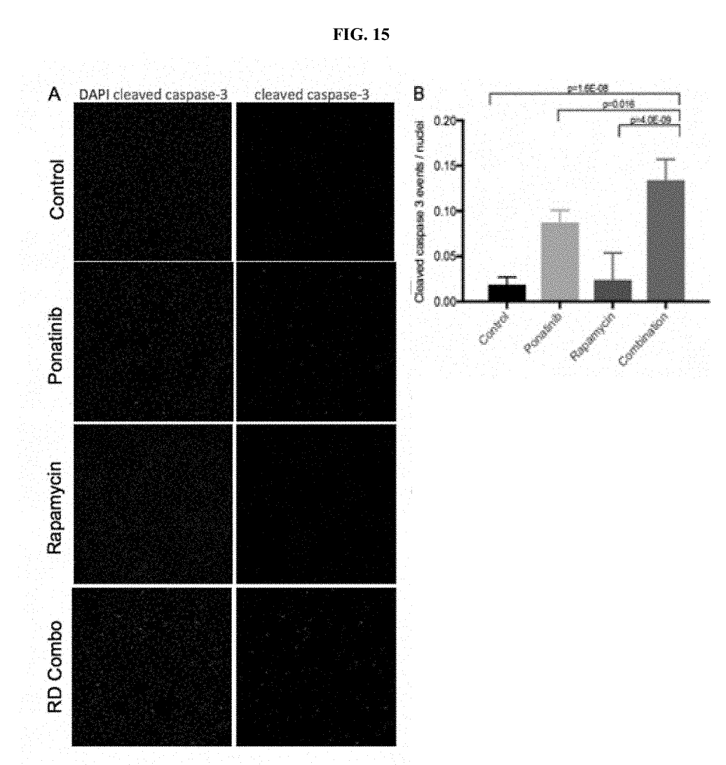

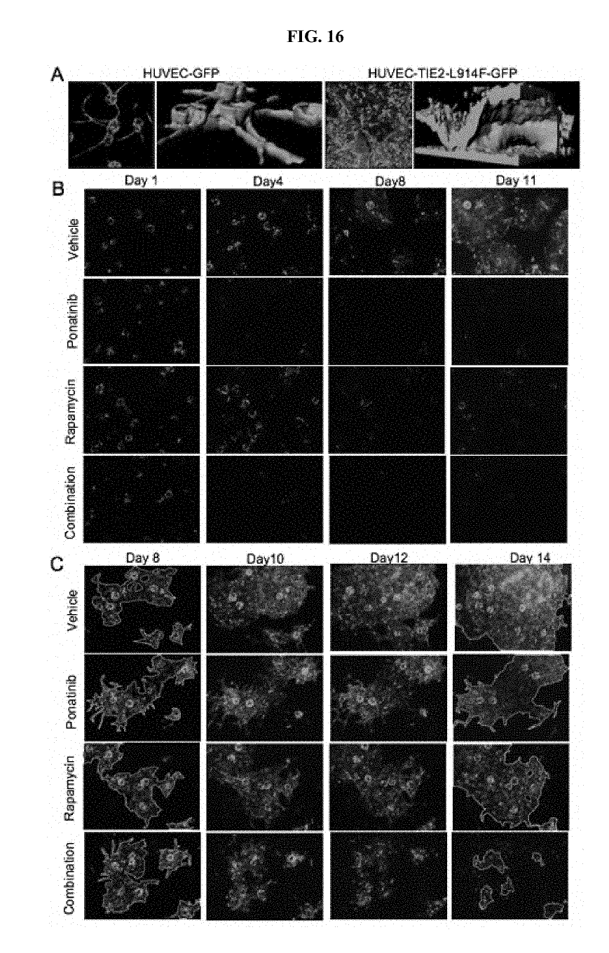

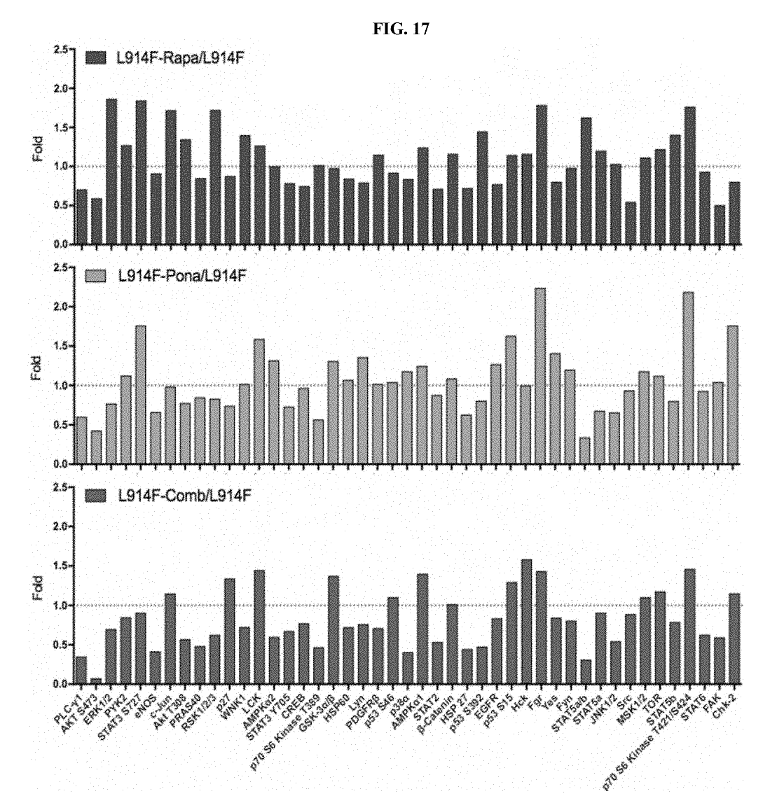

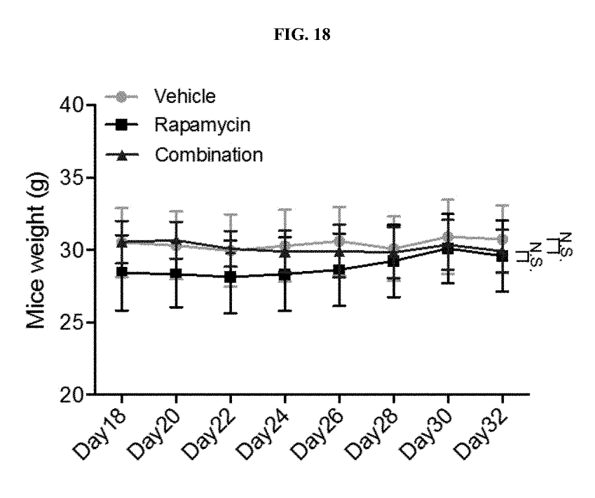

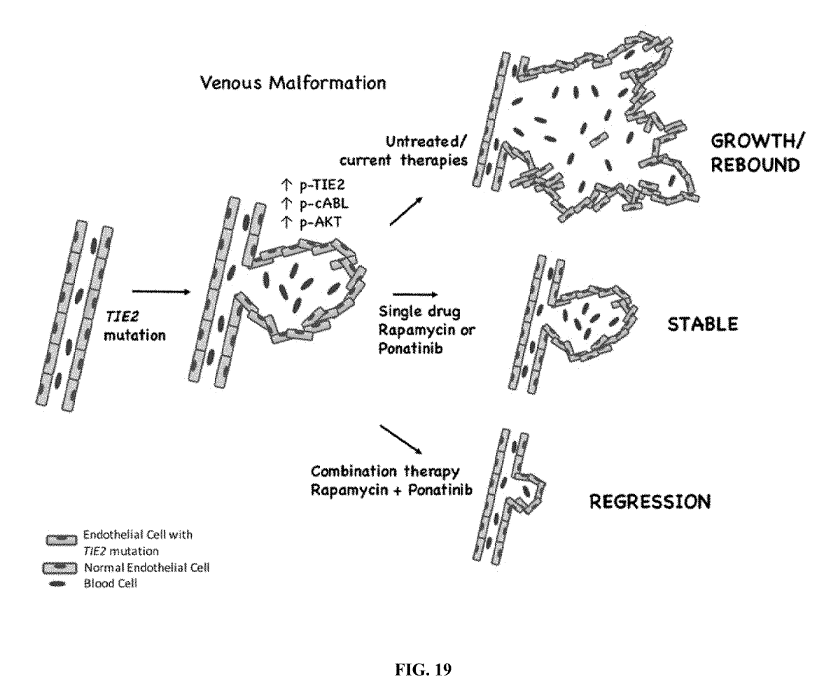

Applicant also evaluated the efficacy of the drug combination in inducing existing tubes to regress. In this experiment compounds were added starting from day 8, when the HUVEC-TIE2-L914F-derived tube network were already established (FIG. 6C). In the vehicle group, the vascular/tube area expanded from day 8 to 14 by 54.50±3.81% while there was only an area expansion of 14.79±4.53% and 20.25±3.23% in Ponatinib and rapamycin single treatment, respectively (FIG. 6D and FIG. 8C). Conversely, in the combination treatment the vascular/tube area regressed by 20.80±2.23%. These data show that combination of Ponatinib with rapamycin induced significant HUVEC-TIE2-L914F-derived vascular network regression, which could be responsible for the lesion reduction recorded in Applicant's in vivo experiments.

Combination of Ponatinib and Rapamycin Enhances AKT Inhibition in HUVEC-TIE2-L914F

To identify signaling networks that are critical for combination treatment response, Applicant analyzed the phosphorylation level of 45 tyrosine kinase sites in HUVEC-TIE2-WT and HUVEC-TIE2-L914F. As shown in FIG. 7A, relative to HUVEC-TIE2-WT, phosphorylation of AKT (both Ser473 and Thr308) and its downstream effectors such as eNOS and PRAS40 was elevated in HUVEC-TIE2-L914F. Besides that, activity of PLCγ1 and ERK along with its substrate RSK1/2/3 were also increased. These results are consistent with previous study that PI3K and MAPK are two main downstream signaling pathways under TIE2 activation(35). Applicant further analyzed phosphorylation changes after 48 hours of treatment with rapamycin, Ponatinib or combination in HUVEC-TIE2-L914F. The drug combination decreased the phosphorylation levels of the most activated kinases in the HUVEC-TIE2-L914F (FIG. 7B). The rapamycin treatment decreased phosphorylation of PLCγ1, AKT (Ser473) and its substrates eNOS and PRAS40, but rapamycin treatment elevated activity of ERK-RSK1/2/3, STAT3 (Ser727), c-Jun and AKT (Thr308). These data were consistent with previous studies that rapamycin treatment increases AKT activation at Thr308 by S6K-PI3K-PDK1 feedback loop and over-activation of ERK(36, 37). Meanwhile, when combining rapamycin with Ponatinib, treatment not only inhibited PLCγ1 and AKT (Ser473), but also suppressed ERK-RSK1/2/3.

Applicant examined these kinase activity changes by Western blotting. HUVEC-TIE2-L914F cells were treated with vehicle, rapamycin, Ponatinib or combination for 48 and 72 hours (FIG. 7C). Ponatinib decreased phosphorylation of TIE2 and ABL in the TIE2 mutant cells (also seen in FIG. 1B), while rapamycin has no effect on these targets. Activity of PLCγ1 was high under rapamycin treatment, but was completely inhibited by Ponatinib. Rapamycin or Ponatinib inhibited AKT activity (Ser437 and Thr308) while drug combination enhanced this inhibition. In addition, drug combination reduced ERK phosphorylation, whose levels tend to increase upon rapamycin treatment.

Combination of Ponatinib and Rapamycin Induces VM Lesion Regression in a PDX Model

Patient-derived xenograft (PDX) models provide translational implications for clinical trials. Here, Applicant established a novel VM-PDX model based on cells isolated from patients and investigated the efficacy of the drug combination. Endothelial cells (VM-EC) were collected from freshly resected patient VM lesion and pooled by CD31-conjugated magnetic beads. VM-EC exhibited endothelial cell morphology (cobblestone phenotype) and expressed endothelial cell specific marker CD31 at the cell membrane (FIG. 8A). Sanger sequencing of VM-EC confirmed single nucleotide substitution of C to T at 2740 base in the TEK gene (FIG. 8B) which resulted in a leucine-to-phenylalanine substitution at position 914 (L914F mutation).

To assess the efficacy of the drug combination, VM-EC were first injected in nude mice, and, at day 18, treatment was started. At day 32 (FIGS. 8C and 8D), VM-EC formed blood filled lesions in the vehicle treated group while combination treatment induced significant lesion regression compared to vehicle (P=1.11E-05) or rapamycin monotherapy (P=2.52E-04). Furthermore, analysis of lesion vessels showed that VM-EC formed blood filled big channels while combination treatment markedly induced vessel regression (FIGS. 8E and 8F). These results suggest that this novel PDX model can be used as a platform to test therapeutic potency of drug treatments. Furthermore, Applicant confirmed the efficacy of Ponatinib combined with rapamycin in inducing regression of VM lesions in this PDX model.

Discussion

To date, there are no targeted therapies promoting significant regression of VM lesions. Here, Applicant uncovered that combining Ponatinib with the mTOR inhibitor rapamycin, even at reduced dose, induces significant murine VM lesion regression and shows well tolerated side effects. Applicant determined that c-ABL is highly phosphorylated in HUVEC-TIE2-L914F and Ponatinib, an ABL kinase inhibitor which targets both ABL and TIE2, significantly suppresses HUVEC-TIE2-L914F proliferation. Furthermore, in vitro, Ponatinib combined with rapamycin has a synergetic effect on TIE2-L914F-HUVEC proliferation and apoptosis, resulting in vascular tube regression. The main molecular mechanisms of the drug combination TIE2-L914F-HUVEC are concomitant AKT (S473) and ERK-RSK1/2/3 inhibition.

ABL kinases are involved in the regulation of multiple cellular functions such as cell proliferation, membrane and organelle trafficking, and cytoskeletal dynamics (25). In endothelial cells (ECs), ABL kinase is phosphorylated upon Angpt1 stimulation to mediate Angpt1/Tie2 signaling that supports EC survival and vascular stability (38). Specific Abl/Arg double knockout in mouse induced late-stage embryonic and perinatal lethality with hepatic necrosis, localized loss of vasculature, and hemorrhage. Other reports demonstrated that ABL kinase activity is required to maintain endothelial cell permeability(39) and normal vascular development(40). Applicant's results showed efficacy of three ABL kinase inhibitors (Ponatinib, Nilotinib and Bosutinib) in decreasing HUVEC-TIE2-L914F proliferation. These results further allowed us to confirm that c-ABL is a major downstream effector upregulated by ligand-independent activated TIE2 (L914F mutation). Applicant's study focused on the most frequent mutation found in VM patients, TIE2-L914F. Whether other somatic or inherited TIE2 mutations could induced ABL activation needs further investigation. In addition, the role of c-ABL activation and its downstream effectors in vascular dysmorphogenesis are still unknown.

Rapamycin (Sirolimus) prevented lesion expansion in HUVEC-TIE2-L914F-derived VM lesions and in a pilot clinical trial(13). In Applicant's in vitro drug screening, two rapamycin analogs, Temsirolimus (Torisel®) and Everolimus (Afinitor®) showed the same effectiveness as rapamycin in the proliferation inhibition of HUVEC-TIE2-L914F. Recently, somatic activating mutations in PIK3CA, which encodes the P110α catalytic subunit, were identified in VM patient samples(15). HUVEC transfected to express these PIK3CA mutations showed levels of AKT phosphorylation comparable to HUVEC-TIE2-L914F, but did not show increased ERK activity. Furthermore, two different Pik3ca^{H1047R} knock-in murine system resulted in formation of venous slow-flow lesions akin to patient VM(10, 41). In the mosaic mesodermal Pik3ca^{H1047R} model, rapamycin treatment was shown to induce a 25% decrease in lesion size, supporting the use of rapamycin for venous malformations caused by PIK3CA mutations. In this study, regression was achieved with 4 mg/kg rapamycin after 4 weeks of treatment, this concentration is higher to the one utilized in Applicant's study and is also higher to the one published in clinical studies (0.8 mg/m²)(6, 7, 42). In the ubiquitously inducible CAG-CreER Pik3ca^{H1047R} model, the PIKα inhibitor BYL719 strongly inhibited VM lesion growth and increased the number of cleaved caspase-3 positive cells, whereas Everolimus prevented lesion growth but did not induce cell apoptosis. Moreover, it was not established if BYL719 was more potent than rapamycin as the two drugs were not analyzed side-by-side.

Taken together, these data highlight the PI3K-AKT-mTOR signaling pathway as a putative target for VM treatment. Applicant show here that in HUVEC-TIE2-L914F rapamycin treatment inhibited AKT phosphorylation and promoted cell cycle arrest and tube formation inhibition. Rapamycin treatment, though, failed to induce significant cell apoptosis and VM lesion regression. Combining rapamycin with Ponatinib enhanced inhibition of AKT (Ser473) and suppressed ERK to induce synergetic proliferation inhibition and murine VM lesion regression. These results suggest that enhanced suppression of PI3K and ERK exerted by the drug combination is an effective therapeutic strategy to improve VMs treatment, especially in TIE2-L914F derived VMs. Notably, Applicant's data in the PDX model further endorse the therapeutical benefits of the drug combination. The future use of PDX models of VM will allow to test the efficacy of the drug combination for VM patient that express mutations different from TIE2-L914F, such as other TIE2 or PIK3CA mutations. Furthermore, patient cells with such mutations should be analyzed in vitro to screen for drugs or compounds effective in targeting the specific mutated protein/s, thus enabling the development of personalized treatment options.

In Applicant's drug screening Ponatinib exhibited the strongest proliferation inhibition alone and in combination with rapamycin compared to other ABL kinase inhibitors. 45 mg Ponatinib versus Imatinib for CML showed earlier molecular responses but an increased risk for vascular occlusive events (43). Two ongoing studies are investigating the effects of 30 and 15 mg daily starting doses (NCT02467270 and NCT02627677). Analysis of different trials comparing Imatinib with Dasatinib, Nilotinib, Ponatinib, or Bosutinib, determined that the risk of vascular occlusive events was increased with all ABL kinase inhibitors, except Imatinib and Bosutinib(44). In Applicant's in vivo studies, Applicant did not record significant side effects or animal deaths in response to Ponatinib or combination treatment.

Intermittent administration of Ponatinib into rapamycin treatment regimen may also induce VM regression without major side effects. Moreover, given that most VMs form slow-flow, localized cutaneous lesions, together with the clinical therapy experience of sclerotherapy, combination of rapamycin and Ponatinib may be optimized by direct lesional injection to reduce systemic toxicity.

In conclusion, to the best of Applicant's knowledge this is the first study reporting the efficacy of Ponatinib combined with rapamycin and this targeted combination therapy induced significant murine VM lesion regression with minimal adverse effects. These findings have implications for future translational studies to meet the imminent needs for VM clinical therapy.

Methods

Reagents

FDA approved drug library was provided by the NCI Development Therapeutics Program (https://dtp.cancer.gov/organization/dscb/obtaining/available_plates.htm). Ponatinib and rapamycin tested in vivo were purchased from LC laboratories.

Cell Culture

HUVECs and retrovirally-transfected HUVECs expressing full-length TIE2-WT or TIE2-L914F were previously described (31). They were expanded in culture on 1% (w/v) gelatin/PBS-coated plates and fed with Endothelial Cell Growth Medium (Lonza)/10% fetal bovine serum (HyClone).

Cell Proliferation Assay and Combination Index (CI) Calculation

Cell proliferation was measured by standard sulforhodamine B (SRB) assays and the optical density (OD) value was read at 540 nm using SpectraMax i3x Multi-Mode Detection Platform (Molecular Devices). The inhibitory rate of compounds was calculated as (OD540 control-OD540 compound)/OD540 control*100%. Combination index (CI) values were calculated as described (26).

Immunoblotting

Cell were washed by pre-cooled PBS then lysed by RIPA lysis buffer with protease inhibitor and phosphatase inhibitor cocktail. Cell lysates were analyzed by Western blot with antibodies against the following: phospho-ABL1 (Y245), ABL1, phospho-AKT (Ser473 and Thr308), AKT, phospho-4E-BP1, 4E-BP1, phospho-p70S6k, p70S6k, phospho-ERK, ERK, phospho-PLCγ1 (Tyr783) and PLCγ1 (Cell Signaling), phospho-TIE2 (Y1102/1108) (EMD Millipore), TIE2 (Abcam) and β-tubulin (Sigma).

In Vivo Murine Model of VM

2.5×10⁶ HUVEC TIE2-L914F were suspended in Matrigel™ and injected sub-cutaneously (s.c.) on both flanks of 6-7 weeks old male athymic nu/nu mice (n=8 mice with 2 injections/group) as described (42). Animals received oral gavage daily of 200 μl vehicle (Citric buffer pH 2.5; 25 mM—Ethanol 30% (v/v) solution), Ponatinib (30 mg/kg), rapamycin (2 mg/kg), combination of both or reduced dose combination (20 mg/kg Ponatinib+1 mg/kg rapamycin). For lesion expansion studies, daily oral administration by gavage feeding started from day 1, while for lesion regression study, treatment started when average lesion size in each group exceeded 100 or 130 mm². Size of lesions was measured with caliper. Lesions were dissected and weighted at the end of treatment, then were fixed in 10% formalin. After Hematoxylin and Eosin (H&E) staining, 5 images were taken randomly per section, then vascular area (%) was quantified with ImageJ software.

Immunohistochemistry

Paraffin sections of murine VM lesions were stained with cleaved caspase-3 (Cell Signaling) or anti-human Ki-67 (Abcam). This was followed by peroxidase-secondary antibody and staining with DAB peroxidase (HRP) substrate (VECTOR Labs). Nuclei were stained with hematoxylin.

Blood Chemical Profile and CBC

Blood samples were collected and sent to CCHMC Veterinary Laboratory for analysis.

Apoptosis Analysis

HUVEC-TIE2-L914F (3×10̂5/well) were seeded in six-well plates and treated with tested compounds for 72 h. Cells were collected according FITC Annexin V Apoptosis Detection Kit protocol (BioLegend). Briefly, cells were re-suspended in cold (4° C.) binding buffer and incubated for 15 min in the dark at room temperature following addition of 5 ml of Annexin V-FITC and 5 ml of 7-AAD solutions. Flow cytometry analysis was performed using FACSCalibur2 and analyzed with CellQuest Pro software.

Cell Immunostaining

HUVEC-TIE2-L914F were seeded in Millicell® EZ SLIDE 4-well glass slides (EMD Millipore), and were treated by DMSO, Ponatinib, rapamycin or combination for 72 hours. Then slides were staining with AlexaFluor® 647 conjugated cleaved-caspase 3 (Cell signaling) to detect cell apoptosis. Nuclei were stained with 4′,6-diamidino-2-phenylindole (DAPI).

Cell Cycle Analysis

HUVEC-TIE2-L914F (4×10̂5/well) were seeded in six-wells plates and treated with tested compounds for 48 h. Cells were collected and fixed in 70% ice-cold ethanol. Before the analysis, cells were treated with Propidium Iodide Flow Cytometry Kit (Abcam) according manufacture protocol. Then cell cycle distribution and sub-G1 DNA content were measured with FACSCalibur2 and analyzed with ModFit-LT3.0 software (Verity Software House).

Migration Assay

HUVEC-TIE2-L914F (1×10̂6/well) were seeded in six-wells plates until confluence was reached and then treated with hydroxyurea for 4 hours before the scratch was performed. Relative cell migration distances per hour were measured with ImageJ software. Two non-overlapping fields were selected and examined per well.

Fibrin Gel Bead Assay

Assay was performed as described(34). Briefly, coated the beads with HUVEC-GFP or HUVEC-TIE2-L914F-GFP cells (2500 beads with 1×10⁶ cells) and mixed coated beads in fibrinogen solution at a concentration of ˜500 beads/ml. Then 0.625 Units/mL of thrombin and 0.5 ml beads/fibrinogen suspension were added per well in 24-well plate successively. After dotting in the 37° C.-incubator, added 1 ml EGM-2 medium and 20,000 fibroblasts per well on the top. Then beads treated by adding DMSO, Ponatinib, rapamycin or combination in the medium

Human Phospho-Kinase Array

Cells were collected and processed according Proteome Profiler Human Phospho-Kinase Array Kit protocol (R&D systems). Membranes were detected by ChemiDoc™ MP Imaging System and dots were quantified by Image Lab software.

Sanger Sequencing

DNA was extracted from cultured patient VM-ECs using QIAamp DNA Mini Kit (Qiagen) according to manufacturer's protocol and quantified on a Nanodrop 2000c Spectrophotometer (ThermoFisher). Approximately 200 ng of DNA was used for PCR using GoTaq Polymerase Master Mix (Promega) as per manufacturer's instructions. Forward (5′ TGGTGTTGCTAGATGTGTTT) and reverse (5′ TTTTGGCTCAAGTAGTCCAT) primers were used to amplify and assess mutations in exon 17 coding sequence containing TIE2 L914 (Integrated DNA Technologies). Product amplification was confirmed by gel electrophoresis on a 2% agarose gel and further purified by gel excision using QIAquick Gel Extraction Kit (Qiagen) following manufacturer's protocol. Purified PCR product were sequenced at CCHMC DNA Sequencing and Genotyping Core. Electropherogram peak results are visualized using CodonCode Aligner (CodonCode Corporation).

VM PDX Model

VM-EC cells were collected form freshly resected VM lesion and pooled by CD31 magnetic beads After cell expansion, 3.5×10̂6 VM-ECs were suspended in Matrigel™ and injected sub-cutaneously (s.c.) on both flanks of 6-7 weeks old male athymic nu/nu mice as described (42). When most lesion turned to red in appearance and average lesion size in each group exceeded 50 mm², animals received oral gavage daily of 200 μl vehicle (Citric buffer pH 2.5; 25 mM—Ethanol 30% (v/v) solution), rapamycin (2 mg/kg) or combination (30 mg/kg Ponatinib+2 mg/kg rapamycin) for 14 days. Size of lesions (mm²) was measured with caliper. Lesions were dissected and weighted at the end of treatment, then were fixed in 10% formalin. After Hematoxylin and Eosin (H&E) staining, 5 images were taken randomly per section, then vascular area (%) was quantified with ImageJ software.

Statistical Analysis

Data for in vivo and in vitro experiments were expressed as means±SD and analyzed by Student's t-test after normal distribution was assessed. In lesion regression experiment, to better fit normality assumptions, the log 10 of size increase and the log 10 of weight were used. All calculations were done by SAS version 9.3. For each group studied: n=8 animals with 2 lesions on each side. Differences were considered significant at p values <0.05.

### REFERENCES AND NOTES

1. L. M. Boon et al., Assignment of a locus for dominantly inherited venous malformations to chromosome 9p. Human Molecular Genetics 3, 1583-1587 (1994).
2. J. Soblet, N. Limaye, M. Uebelhoer, L. Boon, M. Vikkula, Variable somatic TIE2 mutations in half of sporadic venous malformations. Molecular syndromology 4, 179-183 (2013).
3. S. Behravesh et al., Venous malformations: clinical diagnosis and treatment. Cardiovasc Diagn Ther 6, 557-569 (2016).
4. A. Dompmartin, M. Vikkula, L. M. Boon, Venous malformation: update on aetiopathogenesis, diagnosis and management. Phlebology 25, 224-235 (2010).
5. M. Vikkula et al., Vascular dysmorphogenesis caused by an activating mutation in the receptor tyrosine kinase TIE2. Cell 87, 1181-1190 (1996).
6. A. M. Hammill et al., Sirolimus for the treatment of complicated vascular anomalies in children. Pediatric blood & cancer 57, 1018-1024 (2011).
7. D. M. Adams et al., Efficacy and safety of sirolimus in the treatment of complicated vascular anomalies. Pediatrics 137, 1-10 (2016).
8. S. E. Horbach, M. M. Lokhorst, P. Saeed, A. Rothova, C. M. van der Horst, Sclerotherapy for low-flow vascular malformations of the head and neck: A systematic review of sclerosing agents. Journal of Plastic, Reconstructive & Aesthetic Surgery 69, 295-304 (2016).
9. M. Wassef et al., Vascular anomalies classification: recommendations from the International Society for the Study of Vascular Anomalies. Pediatrics 136, e203-e214 (2015).
10. S. D. Castillo et al., Somatic activating mutations in Pik3ca cause sporadic venous malformations in mice and humans. Sci Transl Med 8, 332ra343 (2016).
11. M. Natynki et al., Common and specific effects of TIE2 mutations causing venous malformations. Hum Mol Genet 24, 6374-6389 (2015).
12. M. Uebelhoer et al., Venous malformation-causative TIE2 mutations mediate an AKT-dependent decrease in PDGFB. Human molecular genetics, ddt198 (2013).
13. E. Boscolo et al., Rapamycin improves TIE2-mutated venous malformation in murine model and human subjects. The Journal of clinical investigation 125, 3491-3504 (2015).
14. P. Castel et al., Somatic PIK3CA mutations as a driver of sporadic venous malformations. Sci Transl Med 8, 332ra342 (2016).
15. N. Limaye et al., Somatic Activating PIK3CA Mutations Cause Venous Malformation. Am J Hum Genet 97, 914-921 (2015).
16. Y. Samuels et al., High frequency of mutations of the PIK3CA gene in human cancers. Science 304, 554 (2004).
17. K. C. Kurek et al., Somatic mosaic activating mutations in PIK3CA cause CLOVES syndrome. Am J Hum Genet 90, 1108-1115 (2012).
18. R. A. Maclellan et al., PIK3CA activating mutations in facial infiltrating lipomatosis. Plast Reconstr Surg 133, 12e-19e (2014).
19. J. B. Riviere et al., De novo germline and postzygotic mutations in AKT3, PIK3R2 and PIK3CA cause a spectrum of related megalencephaly syndromes. Nat Genet 44, 934-940 (2012).
20. E. Boscolo et al., AKT hyper-phosphorylation associated with PI3K mutations in lymphatic endothelial cells from a patient with lymphatic malformation. Angiogenesis 18, 151-162 (2015).
21. V. L. Luks et al., Lymphatic and other vascular malformative/overgrowth disorders are caused by somatic mutations in PIK3CA. J Pediatr 166, 1048-1054 e1041-1045 (2015).
22. A. J. Osborn et al., Activating PIK3CA alleles and lymphangiogenic phenotype of lymphatic endothelial cells isolated from lymphatic malformations. Hum Mol Genet 24, 926-938 (2015).
23. D. A. Guertin, D. M. Sabatini, Defining the role of mTOR in cancer. Cancer Cell 12, 9-22 (2007).
24. E. K. Greuber, P. Smith-Pearson, J. Wang, A. M. Pendergast, Role of ABL family kinases in cancer: from leukaemia to solid tumours. Nature Reviews Cancer 13, 559-571 (2013).
25. A. Khatri, J. Wang, A. M. Pendergast, Multifunctional Abl kinases in health and disease. J Cell Sci 129, 9-16 (2016).
26. X. Li, L. J. Tong, J. Ding, L. H. Meng, Systematic combination screening reveals synergism between rapamycin and sunitinib against human lung cancer. Cancer Lett 342, 159-166 (2014).
27. P. J. Houghton et al., Stage 2 combination testing of rapamycin with cytotoxic agents by the Pediatric Preclinical Testing Program. Molecular cancer therapeutics 9, 101-112 (2010).
28. T. O'Hare et al., AP24534, a pan-BCR-ABL inhibitor for chronic myeloid leukemia, potently inhibits the T315I mutant and overcomes mutation-based resistance. Cancer cell 16, 401-412 (2009).
29. T. C. Chou, Drug combination studies and their synergy quantification using the Chou-Talalay method. Cancer Res 70, 440-446 (2010).
30. F. X. McCormack et al., Efficacy and safety of sirolimus in lymphangioleiomyomatosis. N Engl J Med 364, 1595-1606 (2011).
31. E. Boscolo et al., Rapamycin improves TIE2-mutated venous malformation in murine model and human subjects. J Clin Invest 125, 3491-3504 (2015).
32. A. Barilli et al., In human endothelial cells rapamycin causes mTORC2 inhibition and impairs cell viability and function. Cardiovascular Research, (2008).
33. D. C. Fingar et al., mTOR controls cell cycle progression through its cell growth effectors S6K1 and 4E-BP1/eukaryotic translation initiation factor 4E. Molecular and cellular biology 24, 200-216 (2004).
34. M. N. Nakatsu, J. Davis, C. C. Hughes, Optimized fibrin gel bead assay for the study of angiogenesis. JoVE (Journal of Visualized Experiments), e186-e186 (2007).
35. H. Huang, A. Bhat, G. Woodnutt, R. Lappe, Targeting the ANGPT-TIE2 pathway in malignancy. Nat Rev Cancer 10, 575-585 (2010).
36. X. Wan, B. Harkavy, N. Shen, P. Grohar, L. Helman, Rapamycin induces feedback activation of Akt signaling through an IGF-1R-dependent mechanism. Oncogene 26, 1932-1940 (2007).
37. E. Rozengurt, H. P. Soares, J. Sinnet-Smith, Suppression of feedback loops mediated by PI3K/mTOR induces multiple overactivation of compensatory pathways: an unintended consequence leading to drug resistance. Molecular cancer therapeutics 13, 2477-2488 (2014).
38. E. M. Chislock, C. Ring, A. M. Pendergast, Abl kinases are required for vascular function, Tie2 expression, and angiopoietin-1-mediated survival. Proceedings of the National Academy of Sciences 110, 12432-12437 (2013).
39. E. M. Chislock, A. M. Pendergast, Abl family kinases regulate endothelial barrier function in vitro and in mice. PLoS One 8, e85231 (2013).
40. X. Wang et al., Germline mutations in ABL1 cause an autosomal dominant syndrome characterized by congenital heart defects and skeletal malformations. Nat Genet 49, 613-617 (2017).
41. P. Castel et al., Somatic PIK3CA mutations as a driver of sporadic venous malformations. Science translational medicine 8, 332ra342-332ra342 (2016).
42. R. Salloum et al., Response of Blue Rubber Bleb Nevus Syndrome to Sirolimus Treatment. Pediatr Blood Cancer 63, 1911-1914 (2016).
43. J. H. Lipton et al., Ponatinib versus imatinib for newly diagnosed chronic myeloid leukaemia: an international, randomised, open-label, phase 3 trial. Lancet Oncol 17, 612-621 (2016).
44. H. Haguet et al., Risk of arterial and venous occlusive events in chronic myeloid leukemia patients treated with new generation BCR-ABL tyrosine kinase inhibitors: a systematic review and meta-analysis. Expert Opin Drug Saf 16, 5-12 (2017).

What is claimed is:

1. A method of treating venous malformation (VM), comprising the step of administering an mTOR inhibitor and an ABL kinase inhibitor to an individual in need thereof.

2. The method of claim 1, wherein said mTOR inhibitor is selected from rapamycin or pharmaceutically acceptable salt thereof, 42-[3-Hydroxy-2-(hydroxymethyl)-2-methylpropanoate]-rapamycin or pharmaceutically acceptable salt thereof, 42-O-(2-Hydroxyethyl)-rapamycin or pharmaceutically acceptable salt thereof, or a combination thereof.

3. The method of claim 1, wherein said mTOR inhibitor is rapamycin or pharmaceutically acceptable salt thereof.

4. The method of claim 1, wherein said ABL kinase inhibitor is selected from 4-[(4-Methyl-1-piperazinyl)methyl]-N-[4-methyl-3-[[4-(3-pyridinyl)-2-pyrimidinyl]amino]-phenyl]benzamide methanesulfonate or pharmaceutically acceptable salt thereof, 3-(2-imidazo[1,2-b]pyridazin-3-ylethynyl)-4-methyl-N-[4-[(4-methyl-1-piperazinyl)methyl]-3-(trifluoromethyl)phenyl]-benzamide, 4-Methyl-N-[3-(4-methyl-1H-imidazol-1-yl)-5-(trifluoromethyl)phenyl]-3-[[4-(3-pyridinyl)-2-pyrimidinyl]amino]-benzamide, 4-((2,4-Dichloro-5-methoxyphenyl)amino)-6-methoxy-7-(3-(4-methyl-1-piperazinyl)propoxy)3-quinolinecarbonitrile or pharmaceutically acceptable salt thereof, and N-(2-Chloro-6-methylphenyl)-2-[[6-[4-(2-hydroxyethyl)-1-piperazinyl]-2-methyl-4-pyrimidinyl]amino]-5-thiazolecarboxamide or pharmaceutically acceptable salt thereof.

5. The method of claim 1, wherein said mTOR inhibitor and said ABL kinase inhibitor are administered sequentially, or simultaneously, to an individual in need thereof.

6. The method of claim 1, wherein said mTOR inhibitor is administered to said individual daily, and wherein said ABL kinase inhibitor is administered to said individual weekly.

7. The method of claim 6, wherein said mTOR inhibitor is administered at a dose of from about 0.8 mg/m2, and said ABL Kinase inhibitor is administered at a dose of from about 15 mg/m2 to about 45 mg/m2.

8. The method of claim 1, wherein said rapamycin is administered at a dose of from about 3 mg/m2/day to about 6 mg/m2/day, and wherein said ABL kindase inhibitor is administered at a dose of from about 30 to about 90 mg/m2 daily.

9. The method of claim 1, wherein said mTOR inhibitor and said ABL kinase inhibitor are administered in a single composition.

10. The method of claim 1, wherein said mTOR inhibitor and said ABL kinase inhibitor are administered in an amount sufficient to promote cell apoptosis in a venous malformation lesion and/or promote vascular channel regression.

11. The method of claim 1, wherein said mTOR inhibitor and said ABL kinase inhibitor are administered in an amount and for a duration sufficient to reduce a venous malformation lesion size and/or weight.

12. The method of claim 1, wherein said mTOR inhibitor and said ABL kinase inhibitor are administered in an amount and for a duration sufficient to reduce ki-67 expressing proliferative cells as compared to pre-treatment levels of ki-67 expressing proliferative cells.

13. The method of claim 1, wherein said venous malformation is inherited cutaneomucosal venous malformation (VMCM) or blue rubber bleb nevus syndrome (BRBNS).

14. A method of promoting VM lesion regression and/or reducing VM lesion expansion in an individual in need thereof, comprising the step of administering an mTOR inhibitor and an ABL kinase inhibitor.

15. The method of claim 14, wherein said mTOR inhibitor and said ABL kinase inhibitor are administered orally, intravenously, intralesionally, or a combination thereof.

16. The method of claim 14, wherein said mTOR inhibitor and said ABL kinase inhibitor are administered by direct lesional injection.

17. A method of normalizing hemoglobin levels and erythrocyte number in an individual having VM lesions, comprising the step of administering an mTOR inhibitor and an ABL kinase inhibitor.

18. An article of manufacture comprising:
a. a container comprising a label; and
b. a first composition comprising an mTOR inhibitor or a pharmaceutically acceptable salt thereof and a pharmaceutically acceptable carrier;
c. a second composition comprising an ABL kinase inhibitor or a pharmaceutically acceptable salt thereof and a pharmaceutically acceptable carrier;
wherein the label indicates that said first composition and said second composition are to be administered to an individual having or at risk of developing VM lesions.

19. The article of manufacture of claim 18, wherein said first and second composition are provided as a single composition.

20. The article of manufacture of claim 18, further comprising a means for delivery of said composition to an individual.

21. A composition comprising an mTOR inhibitor or a pharmaceutically acceptable salt thereof, an ABL kinase inhibitor or a pharmaceutically acceptable salt thereof, and a pharmaceutically acceptable carrier.


SEQUENCE LISTING (2 sequences)

SEQ ID NO: 1 - DNA, 20 bp, Artificial Sequence
  Forward Primer
tggtgttgctagatgtgttt

SEQ ID NO: 2 - DNA, 20 bp, Artificial Sequence
  Reverse Primer
ttttggctcaagtagtccat